(19) United States
(12) Patent Application Publication
(10) Pub. No.: US 2011/0171309 A1
(43) Pub. Date: Jul. 14, 2011

(54) Compositions and Methods for Composite Nanoparticle Hydrogels

(75) Inventors: Kytai Troung Nguyen, Grand Prairie, TX (US); Prsnesh B. Aswath, Grapevine, TX (US); Abhimanyo R. Sabnis, Durham, NC (US); Aniket S. Wadajkar, Rrlington, TX (US)

(73) Assignee: Board of Regents, The University of Texas System, Austin, TX (US)

(21) Appl. No.: 12/937,232

(22) PCT Filed: Mar. 25, 2009
(86) PCT No.: PCT/US09/38248

§ 371 Date: Mar. 25, 2011

Related U.S. Application Data
(60) Provisional application No. 61/044,041, filed on Apr. 10, 2008.

(51) Int. Cl. A61K 9/14; A61K 47/32

(52) U.S. Cl. 424/487; 514/772.4

(57) ABSTRACT

Provided herein are systems, methods, and compositions for composite nanoparticle hydrogel networks and systems responsive to a first temperature.

### BACKGROUND OF THE INVENTION

The present invention relates generally to hydrogels, and more specifically to nanoparticle hydrogels. Photo cross-linking hydrogels, incorporated with therapeutic agents, have been investigated extensively as drug delivery systems since the major benefit of these hydrogels is that they can be formed in-situ at a specific site by photopolymerization. Various photopolymerizable polymers have been studied, including (di)methacrylic or (di)acrylic derivatives of poly(ethylene glycol) (PEG), poly(ethylene oxide), poly(vinyl alcohol), and diethyl fumarate/poly(propylene fumarate). Of the photo cross-linking hydrogels, poly(ethylene glycol) (PEG)-based materials are widely investigated for biomedical applications due to their advantageous properties such as biocompatibility and low immunogenicity. PEG functionalized with diacrylate (PEGDA) or dimethacrylate groups cross-link to form nondegradable hydrogels that are used in various biomedical applications such as the microencapsulation of islets, controlled release vehicles, adhesion prevention barriers, and bone restorations.

In addition to photo cross-linking hydrogels, environmentally responsive drug delivery systems have also been investigated for controlled drug delivery applications. Such stimuli-responsive systems undergo phase transitions in response to changes in ionic strength, pH, light, electric field, irradiation, or temperature. In particular, among the temperature-sensitive hydrogels reported to date, poly(N-isopropylacrylamide) (PNIPA) and its copolymers have been widely used for pharmaceutical and tissue engineering applications because of their thermal properties. For example, the release of drugs embedded in these hydrogels can be controlled by changing the local temperature. The unique property of PNIPA to undergo a reversible phase transition at temperatures close to body temperature makes it desirable for biomedical applications. This phase transition occurs in aqueous solutions at a lower critical solution temperature (LCST) around 32° C. for PNIPA. At temperatures below the LCST, PNIPA exhibits hydrophilic properties and exists in an individual chain with a coil conformation. Above the LCST, hydrophobic attractions become more favorable, resulting in a sharp transition from the coil to globule conformation, leading to the collapse of the structure to release drugs from the material. The LCST of PNIPA can be further increased to normal body temperature by copolymerizing with hydrophilic monomers such as PEG and acrylamide (AAm).

### SUMMARY OF THE INVENTION

Provided herein are systems, methods and compositions for composite nanoparticle hydrogel systems and networks. The methods, systems, and compositions are set forth in part in the description which follows, and in part will be obvious from the description, or can be learned by practice of the methods, compositions, and systems.

### BRIEF DESCRIPTION OF THE FIGURES

FIG. 1 is a schematic of the photopolymerizable thermoresponsive composite nanoparticle hydrogel system.

FIG. 2A is a photograph of identical composite nanoparticle hydrogels incubated at 23° C. (left) and 40° C. (right); and FIG. 2B is an SEM image of composite nanoparticle hydrogel network with 20% (w/v) PNIPA-AAm nanoparticles, where the hydrogel is swollen.

FIG. 3A is a graph of the main effects of four parameters, N-isopropylacrylamide (NIPA), N,N′-methylenebisacrylamide (BIS), sodium dodecyl sulfate (SDS), and potassium persulfate (KPS), on PNIPA-AAm nanoparticle sizes, where a positive number indicates that the particular parameter had an increasing effect on the particle size as the value changed from a low level (−) to a high level (+) as shown in Table 1, whereas a negative number demonstrates a decreasing effect; and FIG. 3B is a graph of the effects of NIPA and SDS concentrations on PNIPA-AAm nanoparticle sizes, where the asterisk (*) designates samples with particle sizes that are statistically significantly higher than those of the controls, p<0.001 (n=4), and controls were samples with 1.5% NIPA, 0.03% BIS, 0.06% KPS, and 0.044% SDS.

FIG. 4A is a graph of the size distribution of PNIPA-AAm nanoparticles in the size range of 100 nm, 500 nm, and micron-size particles; and FIG. 4B is an SEM image of 100-nm PNIPA-AAm nanoparticles; and FIGS. 4C-E are TEM images of the 100, 300, and 500 nm PNIPA-AAm nanoparticles, respectively.

FIG. 5A is an SEM image of the composite hydrogels composed of 5% PNIPA-AAm nanoparticles; and FIG. 5B is an SEM image of the composite nanoparticle hydrogels composed of 20% PNIPA-AAm nanoparticles.

FIGS. 6A-D are graphs of the protein release profiles for different composite hydrogels formulated as shown in Table 4, where FIG. 6A includes Runs 1 and 5, FIG. 6B includes Runs 2 and 6, FIG. 6C includes Runs 3 and 7, and FIG. 6D includes Runs 4 and 8.

FIGS. 7A, 7C, 7E, and 7H are half-normal plot showing the effect of factors on the (FIG. 7A) protein release rate in phase I (burst release), (FIG. 7C) protein release rate in phase II (sustained burst release), (FIG. 7E) protein release rate in phase III (plateau release), and (FIG. 7H) thermoresponsiveness of the hydrogels (up to 8 hrs); and FIGS. 7B, 7D, 7F, 7G, and 7I are 3D surface plot showing the effect of factors on the (FIG. 7B) protein release rate in phase I (burst release), (7 FIG. D) protein release rate in phase II (sustained burst release), (FIGS. 7F, 7G) protein release rate in phase III (plateau release), and (FIG. 7I) thermoresponsiveness of the hydrogels (up to 8 hrs)

FIG. 8A is a half-normal plot and FIG. 8B is a 3D surface plot showing the effect of the processing factors on the hydrogel swelling ratio.

FIG. 9A is a schematic of the structure of the single layer composite nanoparticle hydrogel; FIG. 9B is a schematic of the structure of the double layer composite nanoparticle hydrogel; and FIG. 9C is a graph of the protein release profiles of single layer (SL) and double layer (DL) hydrogels at 23° C. and 40° C.

FIG. 10 is a graph of the effects of varying durations (0, 1, 3, and 5 min) of long wave, 365-nm UV-light exposure at about 10 mW/cm² on the survival of human aortic smooth muscle cells, in the absence of a UV photoinitiator, and the controls were cells not exposed to UV light (0 min of exposure).

FIG. 11 is a graph of the effects of photoinitiator (Irgacure 2959) concentrations on the survival of human aortic smooth muscle cells, in absence of UV light.

FIG. 12 is a graph of the combined effects of photoinitiator concentrations at four concentrations 0.01%, 0.015%, 0.04%, and 0.08% (w/v)) and UV exposure (with three durations 1, 3, and 5 min) on the survival of human aortic smooth muscle cells.

FIG. 13 is a graph of the effects of ascorbic acid on HASMC cell survival when exposed to 0.15% (w/v) Irgacure 2959 and 5 min of UV exposure, where controls were not exposed to the photoinitiator and UV light, * is a significant difference of p<0.05; and ** is a significant difference of p<0.05 with respect to 0 mg/L ascorbic acid concentration.

FIG. 14 is a graph of the effects of photopolymerized composite hydrogels on HASMC cell survival. Controls were not exposed to the photopolymerized hydrogels; where * represents a significant difference (p<0.05).

### DETAILED DESCRIPTION OF THE PREFERRED EMBODIMENTS

Generally speaking, the composite nanoparticle hydrogel network 10 releases proteins or drugs in a temperature-responsive manner by combining both photopolymerizable and thermoresponsive hydrogels. The composite nanoparticle hydrogel network can be formed in situ at a specific location in the presence of ultraviolet light and a photoinitiator. The composite nanoparticle hydrogel network comprises a plurality of temperature sensitive poly(N-isopropylacrylamide-co-acrylamide) (PNIPA-AAm) nanoparticles uniformly embedded in a cross-linked hydrogel network. In one embodiment, the hydrogel network is a poly(ethylene glycol) diacrylate (PEGDA), alternatively, the hydrogel network is poly(ethylene glycol) poly(D,L-lactide) (PEG-PLA), poly(ethylene glycol) hyaluronic acid (PEG-HA), or poly(ethylene glycol) poly(glycolic acid) (PEG-PGA). A drug release from the hydrogel network can be controlled by changing the temperature locally at or above a lower critical solution temperature (LCST) of the PNIPA-AAm nanoparticle, where the nanoparticle hydrophobically collapses and expels water in an entropically favored fashion, i.e. swelling and shrinking events. In one embodiment, the composite nanoparticle hydrogel network may be used as a smart protein/drug delivery system for wound healing applications, where the composite nanoparticle hydrogel network is delivered at the injured or wound site. Alternative applications are described below. The composite nanoparticle hydrogel network includes spatial and temporal control of reaction kinetics, fast curing rates to provide rapid polymerization, and effective control over cross-linking density to govern release rates.

“Network” is the composite nanoparticle hydrogel after photopolymerization. “System” is the components of the composite nanoparticle hydrogel including the precursor solution with photoinitiators and the UV light before the photopolymerization of the composite nanoparticle hydrogel network.

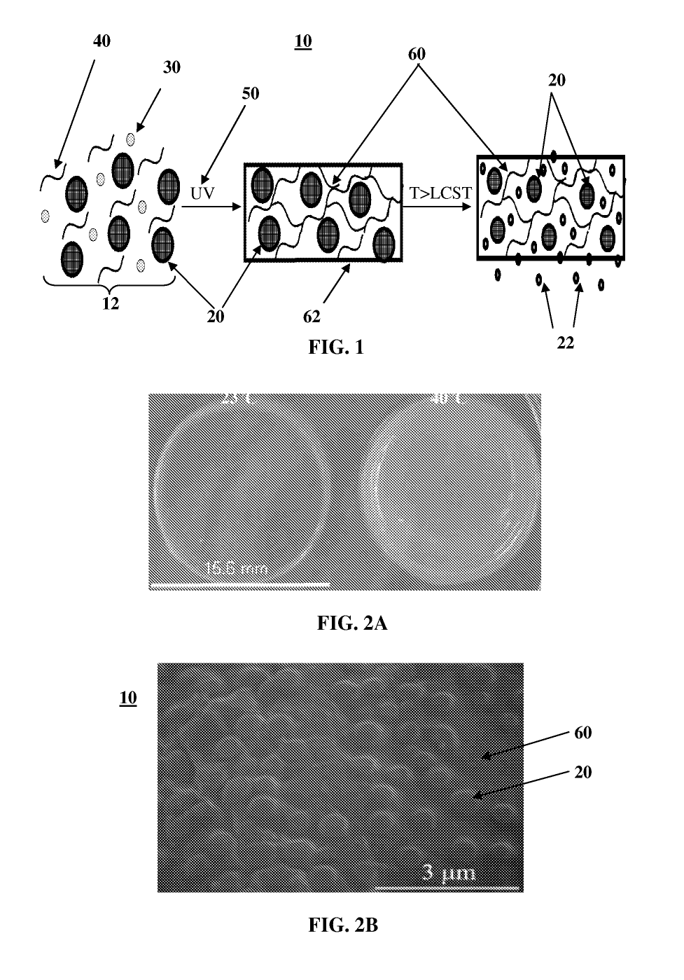

The schematic of the composite nanoparticle hydrogel system 10 is generally shown in FIG. 1, including a precursor solution 12 comprised of the drug- or protein-loaded PNIPA-acrylamide (PNIPA-AAm) nanoparticles 20, a plurality of photoinitiators 30, and a plurality of PEGDA monomers 40. The photoinitiators 30 may include 2,2-dimethoxy-2-phenylacetophenone (Irgacure 651), 1-hydroxycyclohexyl phenyl ketone (Irgacure 184), 2-methyl-1-[4-(methylthio)phenyl]-2-(4-morpholinyl)-1-propanone (Irgacure 907), 2,2′-azobis-(2-amidinopropane)hydrochloride (AAPH), 2,2′-azobis-(2-methyl propionamide)dihydrochloride and 2-hydroxy-1-[4-(hydroxyethoxy)phenyl]-2-methyl-1-propanone (Darocur 2959) and may be selected based upon cytotoxicity. In the presence of ultraviolet (UV) light 50 and the photoinitiators 30, the precursor solution 12 forms a hydrogel network 60 from the PEGDA monomers uniformly entrapping the PNIPA-AAm nanoparticles 20 to form a protective barrier 62. The hydrogel network 60 and the protective barrier 62 may take on any shape or form. The protective barrier 62 may protect the PNIPA-AAm nanoparticles 20 from the reticuloendothelial system (RES), as nanoparticles are often removed by RES. The PNIPA-AAm nanoparticles 20 maintain their morphology and size distribution. The PNIPA-AAm nanoparticles 20 are temperature sensitive and includes a lower critical solution temperature (LCST), such that when the local temperature is increased to or above the LCST, the PNIPA-AAm nanoparticles 20 undergoes a reversible phase transition, collapsing and expelling the drugs or protein 22 into the surrounding tissue. The hydrogel network 60 also includes a second state at a temperature to be in a swollen state allowing drugs or protein 22 released from the PNIPA-AAm nanoparticles 20 to be diffused through the swollen state of the hydrogel network 60. The temperature for the second state of the hydrogel network 60 may be equal or greater than the LCST of the PNIPA-AAm nanoparticles 20, as PEG is hydrophilic, thus the swollen state temperature is increased. A comparison of the one embodiment of the composite nanoparticle hydrogel network 10 maintained at two different temperatures, i.e. 23° C. and 40° C., is shown in FIG. 2A. An SEM image of the composite nanoparticle hydrogel system shows the PNIPA-AAm nanoparticle 20 and the hydrogel network 60 in a swollen state releasing the drugs from the PNIPA-AAm nanoparticle 20 is shown in FIG. 2B.

In one embodiment, the thermoresponsive nanoparticles 20 are loaded with bioactive molecules 22 and then entrapped within the hydrogel network 60 and the LCST is between about 39-40° C., i.e. 39.10, 39.19, 39.90. 39.99, etc. Delivery of bioactive molecules 22 such as proteins, genes, and peptides is one application of the composite nanoparticle hydrogel network 10 compared to other drug delivery carriers, as the composite nanoparticle hydrogel network molecules are easily denatured by extreme heat and organic solvents. The composite nanoparticle hydrogel network 10 includes a hydrogel cross-linking density and a thermoresponsive property of PNIPA-AAm nanoparticles, both which affect the release of such bioactive molecules in a synergistic manner. In one embodiment, the composite nanoparticle hydrogel network includes a cross-linking density and a LCST comprising a triphasic release of the bioactive molecule, where the triphasic release first includes an initial burst release (Phase I), a second sustained burst release (Phase II), and followed by a third plateau release (Phase III). In one embodiment, the initial burst release may comprise the time period within the first hour, the second sustained burst release may comprise from 1 to 8 hours, and the third plateau release may comprise the time period from 8 to 48 hours. While examples below are given for protein release rates, it should be appreciated that other molecules can be substituted for the release rates, according to one of ordinary skill in the art. The nanoparticles may be modified to prolong the drug release from the composite hydrogels, such as by using B-cyclodextrin by forming inclusion complexes with drugs.

The hydrogel network may include degradable segments for the photopolymizable degradable nanoparticle hydrogel networks, such as PEG-HA, PEG-PGA, or PEG-PLA. PEG-PLA includes the biodegradable segment PLA and may be synthesized as follows: 10 g of PEG (average MW 3350) and D,L-lactide were taken in a molar ratio of 1:5 and were placed in a dried round bottom flask and purged with argon for 5 mins. The molecular weight of PEG and PLA may be varied to alter the hydrogel swelling ratio, triphasic release and degradation rate, as detailed below. The contents were stirred and 40 μl of stannous octoate was added as catalyst. The whole system was sealed in inert argon atmosphere and placed in an oil bath which was heated to 140° C. The reaction was allowed to progress for 4 hours. The final product was dissolved in anhydrous dichloromethane and followed by precipitation in ethyl ether stirred at 2000 rpm where the product was slowly dripped into to avoid clumping of the copolymer.

PEG-PLA may be further acrylated with dimethacrylate (DA) by dissolving 5 g of PEG-PLA in 75 mL of anhydrous dichloromethane in a dried round bottom flask and the system was purged with argon for 10 mins to remove any traces of oxygen or moisture. 2.5 mL of triethylamine was added to the dissolved copolymer. 3 mL of acryloyl chloride was dispersed in 25 mL anhydrous dichloromethane and was added drop wise to the copolymer solution using a syringe pump. The reaction was allowed to proceed for 24 hours in an ice bath. The next day, 25 mL of anhydrous dichloromethane was added to the product, and it was filtered through a 0.2 μm filter paper to remove the TEA salt. The final product was then obtained by precipitating the acrylated polymer solution in excess of ethyl ether stirred at 2000 rpm.

PLA-PEG-DA hydrogels may then be photopolymerized by taking the precursor solution consisted of 15%(w/v) PLA-PEG-DA in de-ionized water. Irgacure 2959 was added as the photo initiator to the solution at a concentration of 0.15% (w/v). 200 μL of the hydrogel precursor solution was then allocated into the wells of a 48 well microplate and photopolymerized under a UV lamp for 3 minutes.

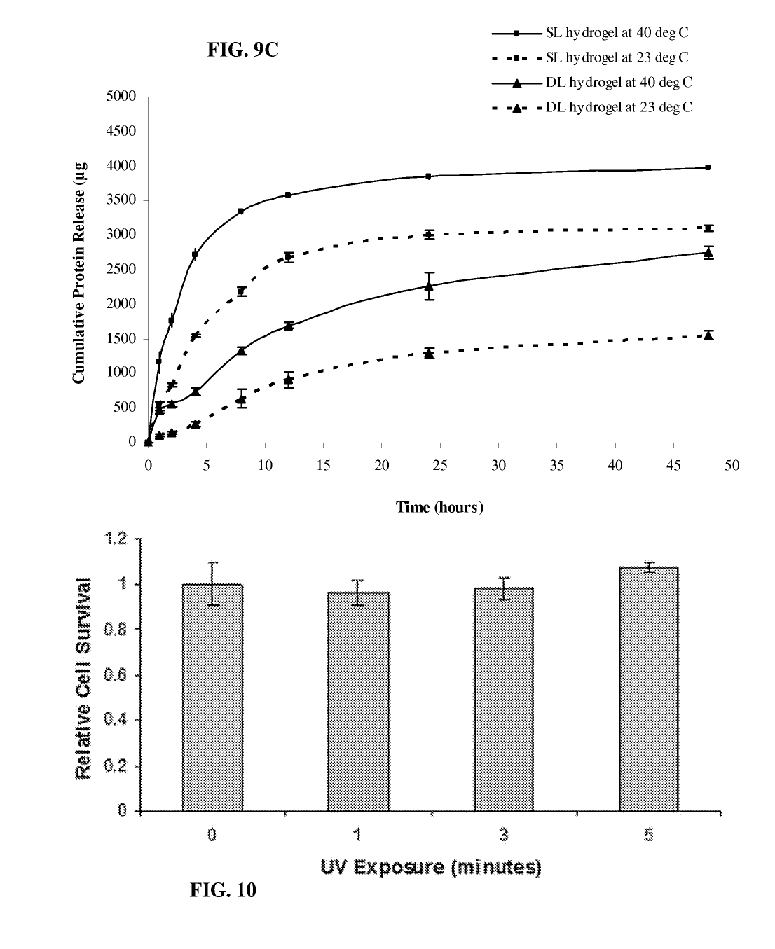

The composite nanoparticle hydrogel network 10 includes a hydrogel swelling ratio and the triphasic release, both which respond to different factors including PEGDA molecular weight, PEGDA concentration, PNIPA-AAm nanoparticle concentration, and temperature. In an alternative embodiment, composite nanoparticle hydrogel network 10 includes a double layer hydrogel network, where with the inner layer of the hydrogel network contains the protein/drug loaded nanoparticles and the outer layer of the hydrogel network does not contain protein/drug loaded nanoparticles, as shown in FIG. 9A. The outer layer of the hydrogel network is another diffusion layer affecting the protein/drug release characteristics of the protein/drug loaded nanoparticles and contributing to a sustained release profile. Alternatively, PNIPA nanoparticles may be embedded in the outer layer of the hydrogel, where the outer layer is embedded with PNIPA nanoparticles loaded with different drugs.

In one embodiment, a factorial analysis evaluates the effects of PEGDA concentration, PEGDA molecular weight, PNIPA-AAm nanoparticle concentration, and temperature on the protein release profiles and swelling ratios of the hydrogel network. Factorial analysis is a statistical method used to describe variability among observed variables in terms of fewer unobserved variables called factors. The observed variables are modeled as linear combinations of the factors, plus “error” terms. The information gained about the interdependencies can be used later to reduce the set of variables in a dataset. Factorial analysis will also detect and quantify special relationships in which two or more factors act differently in how they affect process together compared to how they affect it separately. Since complicated processes take a long time to explore, factorial analysis reduces the time to allow for less experiments. The PNIPA-AAm nanoparticle concentration and temperature may be important factors affecting the protein release during the burst release phase. Additionally, PEGDA molecular weight may be a significant factor affecting the protein release in the plateau region, and an important factor that controls the hydrogel swelling ratio.

For drug loading and release studies Bovine serum albumin (BSA), as a model protein was used. Alternatively, any pharmacologically active or therapeutic agent may be loaded into the PNIPA-AAm nanoparticles selected from the group of antibiotic drugs, antiviral drugs, anti-cancer drugs, chemotherapy drugs, neoplastic agents, steroids, anti-clotting drugs, aspirin, peptides, antiproliferative agents, antioxidants, antimetabolites, non-steroidal and steroidal anti-inflammatory drugs, immunosuppresents, genetic materials including DNA and RNA fragments for gene delivery, antibodies, lymphokines, growth factors, radionuclides, and the like.

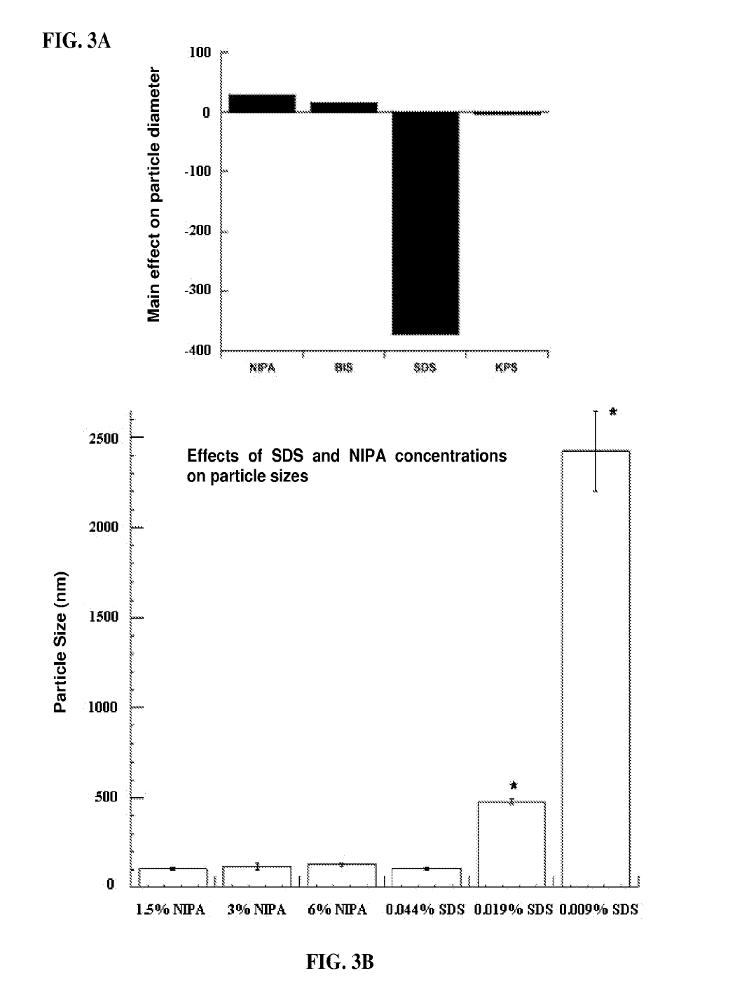

In one embodiment, the PNIPA-AAm nanoparticles 20 may be prepared by adding to an aqueous solution (100 ml) containing N-isopropylacrylamide, acrylamide, crosslinker N,N′-methylenebisacrylamide, sodium dodecyl sulfate (SDS), and stirring under argon gas for 30 minutes. The SDS stabilizes the resultant particles by acting as a surfactant to reduce the size of the PNIPA-AAm nanoparticle prepared. Potassium persulfate (KPS) as the initiator (0.0624 g) is then added and radical polymerization is carried out at 70° C. for 4 hours under argon. In one embodiment, different amounts of NIPA, BIS, SDS, and KPS, may be added in different combinations to result in different size PNIPPA-AAm nanoparticles. The amounts may be added in high (+) ranges for NIPA, BIS, SDS, and KPS, at 6.0, 0.030, 0.044, and 0.06% w/v, respectively in combination with low (−) ranges for NIPA, BIS, SDS, and KPS, at 1.5, 0.015, 0.019, and 0.03% w/v, respectively, as shown in Table 1. Alternatively, the amounts of SDS may be varied to results in different size nanoparticles, as shown in Table 2. The resulting PNIPA-AAm nanoparticles are cooled to room temperature and dialyzed (6-8 kDa MW cutoff) against deionized water for 4 days to remove unreacted monomers and surfactants. The resultant dialyzed PNIPA-AAm nanoparticles are then measured for their sizes using a Beckmann-Coulter Particle Analyzer (model LS230). The effects of these factors on the PNIPA-AAm nanoparticle size are shown in FIG. 3A. The size of PNIPA-AAm nanoparticles was most effected by SDS, whereas varying other parameters showed a minimal effect (FIG. 3A). The effects of two influencing factors, SDS and NIPA, on the nanoparticle size were tested (Table 2), and the results are demonstrated in FIG. 3B. The factorial design analysis indicated that SDS was the most important influencing factor on the size of PNIPA-AAm nanoparticles as shown in FIGS. 3A and 3B. The nanoparticles may include sizes of about 50-500 nm, about 75-250 nm, or about 100 nm. The nanoparticles may include a shape of spherical, cuboidal, and the like.

TABLE 1
Half-Factorial Design To Determine Factors Affecting
the PNIPA-AAm Particle Size and Combinations of the
Experimental Variables in Experiment Design.

| Run | NIPA | BIS | SDS | KPS | Size (nm) |
|---|---|---|---|---|---|
| 1 | + | + | + | + | 134 ± 7.5 |
| 2 | + | + | − | − | 521 ± 12.2 |
| 3 | − | − | − | − | 465 ± 8.3 |
| 4 | − | − | + | + | 103 ± 2.7 |
| 5 | − | + | − | + | 478 ± 17.5 |
| 6 | + | − | + | − | 121 ± 13.5 |
| 7 | + | − | − | + | 489 ± 24.1 |
| 8 | − | + | + | − | 107 ± 5.7 |

TABLE 2
Effects of NIPA and SDS on PNIPA-AAm Particle Sizes

| | |
|---|---|
| Reagents (% w/v) | Particle Size |

| NIPA | BIS | KPS | SDS | (nm) |
|---|---|---|---|---|
| 1.5 | 0.03 | 0.06 | 0.044 | 107 ± 5.7 |
| 1.5 | 0.03 | 0.06 | 0.019 | 478 ± 17.5 |
| 1.5 | 0.03 | 0.06 | 0.009 | 2425 ± 221.2 |
| 3.0 | 0.03 | 0.06 | 0.044 | 121 ± 13.5 |
| 6.0 | 0.03 | 0.06 | 0.044 | 134 ± 7.5 |

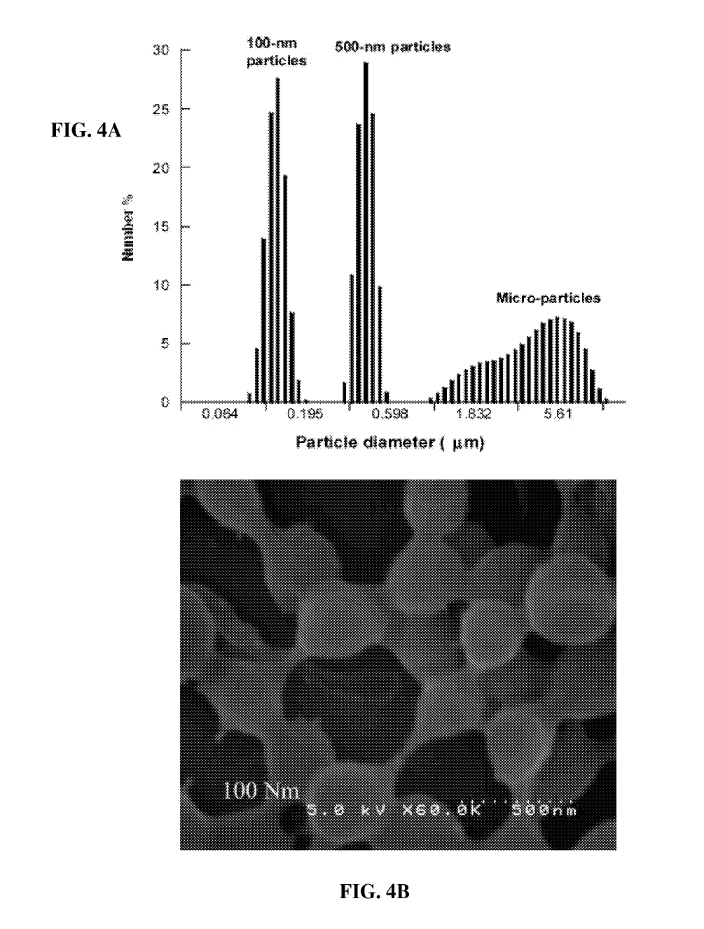

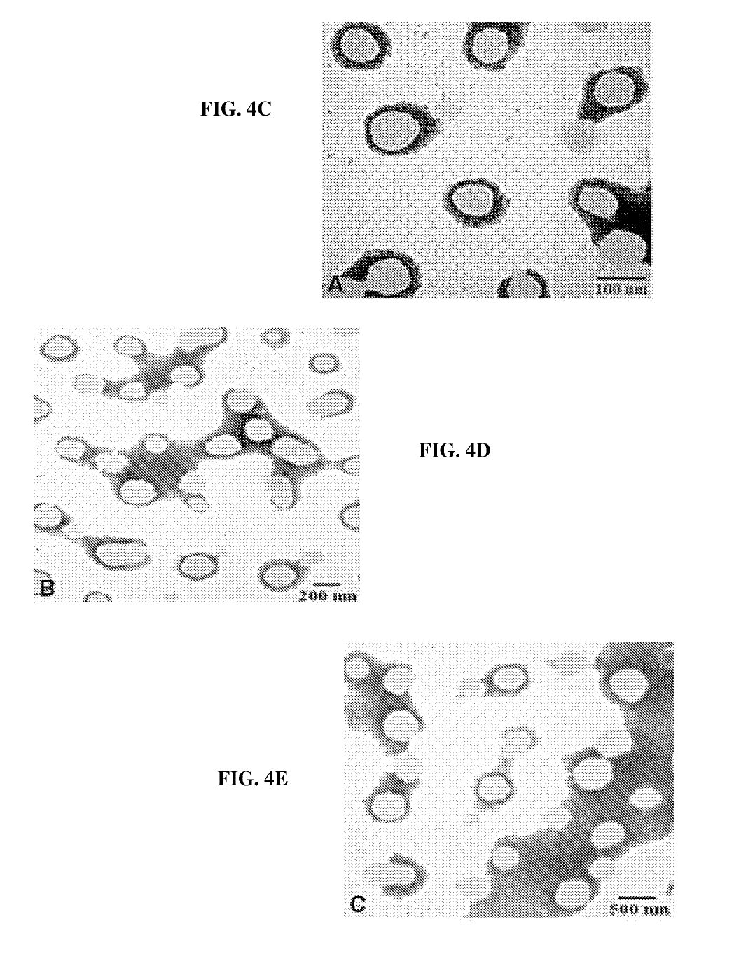

The distribution characteristics of PNIPA-AAm nanoparticles are given in FIG. 4A. The size distribution characteristics are in a narrow range for the smaller sized nanoparticles. More than 80% of the nanoparticles were within the range of 40-200 nm for 100-nm size particles, whereas more than 95% of the particles were found to be within the 400-600 nm range for the 500-nm size nanoparticles, as shown in FIG. 4A. In contrast to the nanoparticles, microparticles have a wide distribution in size, ranging from 1 to 6 μm. Additionally, SEM imaging of the 100-nm PNIPA-AAm nanoparticles may be performed in order to confirm the formation of the PNIPA-AAm nanoparticles after the emulsion polymerization reaction. As shown in FIG. 4B, the PNIPA-AAm nanoparticles are distributed uniformly within the range of 40-200 nm (similar to the results from the particle analyzer), and the surfaces of these particles are spherical. FIGS. 4C-E are TEM images of the 100, 300, and 500 nm PNIPA-AAm nanoparticles, respectively. Transmission electron microscope (TEM, JEOL 1200 EX Model) may also be used to determine the size and shape of the synthesized PNIPA-AAm nanoparticles. In general, samples were prepared by drop casting an aqueous dispersion of nanoparticles onto a carbon coated copper grid. The nanoparticles are stained with phosphotungstic acid (PTA) at a concentration of 0.01 wt. % before observation. Measurements of nanoparticle sizes and size distributions may be performed in de-ionized water using dynamic light scattering technology (Nanotrac 150, Microtrac Inc.). In one embodiment, the 100 nm PNIPA-AAm nanoparticles may be prepared by 26.20 mg of BIS, 43.90 mg of SDS, 62.40 mg of KPS, and 1.54 g of NIPAAm in a total volume of 100 mL of DI water. The 300 nm nanoparticles may be prepared by 26.10 mg of BIS, 30.80 mg of SDS, 60.00 mg of KPS, and 1.50 g of NIPAAm for a total volume of 100 mL. 500 nm nanoparticles may be prepared by 30.00 mg of BIS, 19.00 mg of SDS, 30.00 mg of KPS, and 1.50 g of NIPAAm in a total volume of 100 mL.

PNIPA-AAm nanoparticles are known to be biocompatible, if by chance the PNIPA-AAm nanoparticles are able to migrate out of the hydrogel network. PNIPA-AAm nanoparticle concentrations of 0.1, 1, 5, and 10 mg/mL did not show a significant decrease in cell survival when 3T3 fibroblast cells are exposed to PNIPA-AAm nanoparticles, as disclosed in Wadajkar et al. “Cytotoxic evaluation of N-isopropylacrylamide monomers and temperature sensitive poly(N-isopropylacrylamide) nanoparticles”, J. Nanopart. Res. 26: 3055-3064 (2005), herein incorporated by reference. The cross-linked surface structure of the PNIPA-AAm nanoparticles reduces cytotoxicity.

In one embodiment, the composite nanoparticle hydrogel network is produced with various compositions of PNIPA-AAm nanoparticles (5, 10, and 20% weight/volume) mixed with a buffer solution (50 mM HEPES) containing a UV cross-linker, poly(ethylene glycol) diacrylate (PEGDA) to have a PEG final concentration of 100 mg/mL and a UV photoinitiator, Irgacure-2959 at a final concentration of 5 mg/mL. PEGDA increases the LCST, hydrophilicity, biocompatibility of the system, and enhance and accelerate the detachment of cell lays from the copolymer matrix. The resultant solution (200 μL) is uniformly mixed and then dispensed in a 48-well plate and instantaneously exposed to UV light at a wavelength of 365 nm for approximately 1 min to enable formation of PNIPA-AAm particle composite hydrogels. Uniform distribution of nanoparticles within the hydrogel network is dependent on the mixing process of the precursor solution, such as by using pipettes and vortexing. Equivalent hydrogels without PNIPA-AAm nanoparticles may also obtained to serve as controls.

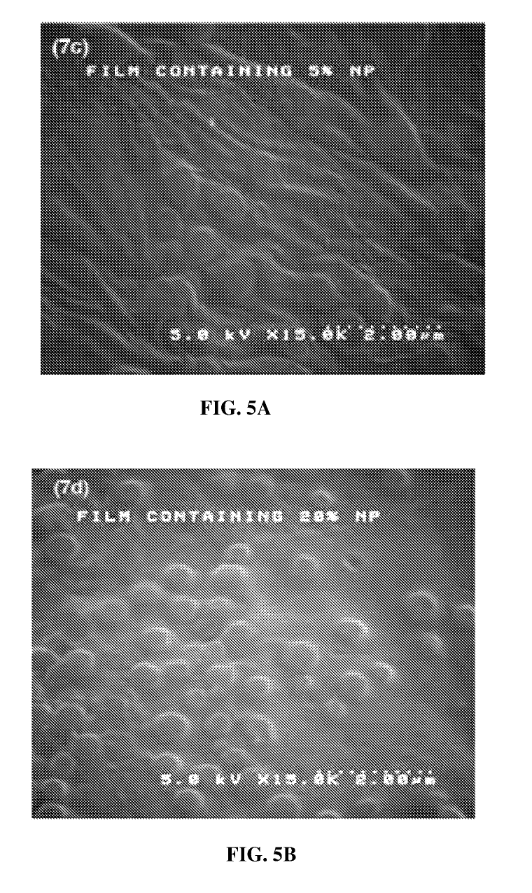

As shown in FIGS. 5A and 5B, SEM images verify the presence of PNIPA-AAm nanoparticles embedded and uniformly distributed in the composite nanoparticle hydrogel network. As shown in FIGS. 5A and 5B, the higher the compositions of nanoparticles used for embedding, the higher the amount of nanoparticles distributed inside the composite nanoparticle hydrogel network. These images also demonstrated that PNIPA-AAm nanoparticles are embedded uniformly inside the PEG polymeric matrix, and the PNIPA-AAm nanoparticles did not change their original size range. SEM measurement of approximately 50 particles within the 20% (w/v) PNIPA-AAm nanoparticles hydrogels (FIG. 5B), shows an average diameter=229±63.3 nm, versus the original PNIPA-AAm nanoparticles (FIG. 4B), with an average diameter=241±33.1 nm, n=50 particles. These PNIPA-AAm nanoparticles also maintained the spherical morphology within the composite matrix (FIGS. 4B, 5A, and 5B).

Alternatively, the evaluation of two molecular weights (MW) (3.4 kDa and 8 kDa) of the cross-linker polymer, PEGDA may be included in a factorial design of the composite nanoparticle hydrogel system. Alternative MW of PEGDA may include about 1 to about 20 kDa. In one embodiment, PEGDA is synthesized by dissolving 12 g of poly(ethylene glycol) (3.4 kDa or 8 kDa) in 36 ml of anhydrous dichloromethane. To the dissolved PEG solution, 1.3 ml of triethylamine is then added and the PEG solution is bubbled with argon gas for 5 minutes. Then 0.61 ml of acryloyl chloride is dissolved in 10 ml of dichloromethane and added drop by drop slowly (over an hour or two) to the flask with bubbled PEG solution. The solution is then stirred under argon for 2 days on an ice bath. The solution is then washed with 2M K₂CO₃ to separate the dichloromethane phase, followed by drying with anhydrous MgSO₄. PEGDA is then precipitated using ethyl ether. Finally, the PEGDA product is filtered and dried for 12 hours under vacuum at room temperature.

The synthesis of the composite nanoparticle hydrogel network 10 may include factorial studies utilized with Design Expert, a Design of Experiments (DOE) software (Stat-Ease Inc., Minneapolis, Minn.), to elucidate the effect of individual and multiple factors on the protein release rate and swelling ratio of the composite nanoparticle hydrogel system. Using DOE, a half-factorial experiment (8 instead of 16 runs) for four factors for the protein release experiments may be designed. The four factors (independent variables) included the PEGDA MW (3.4 kDa and 8 kDa), PEGDA concentration (10% and 15% w/v), PNIPA-AAm nanoparticle concentration (2% and 4% w/v), and temperature (23° C. and 40° C.). For the hydrogel swelling experiments, a half-factorial experiment (4 runs instead of 8 runs) for three factors (where temperature is constant at 23° C.) may be designed. The evaluated responses (dependent outcomes) included the protein release rate and the swelling ratio of the hydrogels. The resulting factorial design is shown in Tables 3, 4, and 5. PEGDA molecular weight, PEGDA concentration, PNIPA-AAm nanoparticle concentration, and temperature are represented as M, P, N, and T, respectively. Alternatively, the PEGDA concentration may range from 5-20%, the PNIPA-AAm nanoparticle concentration may range from 1%-10% w/v, and the temperature may range from 20-50° C. Preferably, the temperature is about 40-45° C., the PNIPA-AAm nanoparticle concentration is about 8%-10%, and the PEGDA concentration is about 10-15% for to form the hydrogels for controlled release.

TABLE 3
Low and high levels for the half factorial design,
where M is the molecular weight of PEGDA, P is the
concentration of PEGDA, N is the concentration of
PNIPA-AAm nanoparticles, and T is the temperature

| Level | M (kDa) | P (% w/v) | N (% w/v) | T (° C.) |
|---|---|---|---|---|
| Low (0) | 3.4 | 10 | 2 | 23 |
| High (1) | 8.0 | 15 | 4 | 40 |

TABLE 4
Actual values of the half 4-factor design; and the protein release
rate in each of the three regions of the single layer hydrogels

| | | | | | Protein Release Rate (μg/hr) | | |
|---|---|---|---|---|---|---|---|
| Run | M | P | N | T | 0-1 | 1-8 | 8-48 |
| # | (kDa) | (% w/v) | (% w/v) | (° C.) | hour | hours | hours |
| 1 | 3.4 | 10 | 2 | 23 | 644 | 185 | 7.3 |
| 2 | 8.0 | 10 | 4 | 23 | 1216 | 264 | 16.3 |
| 3 | 8.0 | 15 | 2 | 23 | 663 | 13 | 13.0 |
| 4 | 3.4 | 15 | 4 | 23 | 603 | 129 | 10.0 |
| 5 | 3.4 | 10 | 2 | 40 | 1222 | 212 | 8.2 |
| 6 | 8.0 | 10 | 4 | 40 | 1927 | 383 | 11.1 |
| 7 | 8.0 | 15 | 2 | 40 | 1128 | 167 | 10.5 |
| 8 | 3.4 | 15 | 4 | 40 | 1818 | 311 | 8.5 |

TABLE 5
Actual values of the half 3-factor design and the swelling ratio

| Run # | M (kDa) | P (% w/v) | N (% w/v) | Swelling Ratio |
|---|---|---|---|---|
| 1 | 3.4 | 10 | 2 | 12.79 |
| 2 | 8.0 | 10 | 4 | 19.05 |
| 3 | 8.0 | 15 | 2 | 17.31 |
| 4 | 3.4 | 15 | 4 | 9.95 |

In one embodiment, the composite nanoparticle hydrogel network 10 may be prepared by dispersing PNIPA-AAm nanoparticles in deionized water to get a stock suspension. Bovine serum albumin (BSA), as a model protein, is then added to the stock suspension at a concentration of 5% (w/v) and incubated at 4° C. for 4 days. Hydrogels (n=4 for each run) for the factorial analysis may be prepared based on Table 4. For example, run 1 is prepared by dissolving 0.1 g of PEGDA (3.4 kDa) in 800 μl of BSA-loaded PNIPA-AAm nanoparticle suspension. 200 μl of the photoinitiator stock solution (0.0125 g/ml) was then added to this solution to make 1 ml of the total precursor solution with final PEGDA and PNIPA-AAm nanoparticle concentrations of 10% (w/v) and 2% (w/v), respectively. To form the hydrogel network, 200 μl of the precursor solution is added to a 48-well plate and exposed to UV light at about 10 mW/cm² for less than 5 minutes. The concentration of the photoinitiator and duration of UV exposure is optimized by evaluating cytotoxic effects of the photoinitiator and UV exposure on fibroblast and smooth muscle cell viability, as detailed in the Examples Section below. 2-hydroxy-1-[4-(2-hydroxyethoxy)phenyl]-2-methyl-1-propanone-1-one (Irgacure 2959) may be used as the photoinitiator.

To evaluate the effect of the factors on protein release, protein loaded nanoparticle hydrogels (n=4) for each run may be incubated at room temperature (23° C.) (below LCST) and at 40° C. (above LCST) in 24-well plates with 1 ml of PBS solution. At the pre-determined time points (1, 2, 4, 8, 12, 24, and 48 hours), the PBS solution from the each well is replaced with 1 ml of fresh PBS solution. The samples collected at various time points are then analyzed using the BCA protein assay (Pierce, following manufacturer's instructions) to evaluate the amount of protein released from the hydrogel. The data is analyzed and the protein release profiles for each run at both temperatures were plotted.

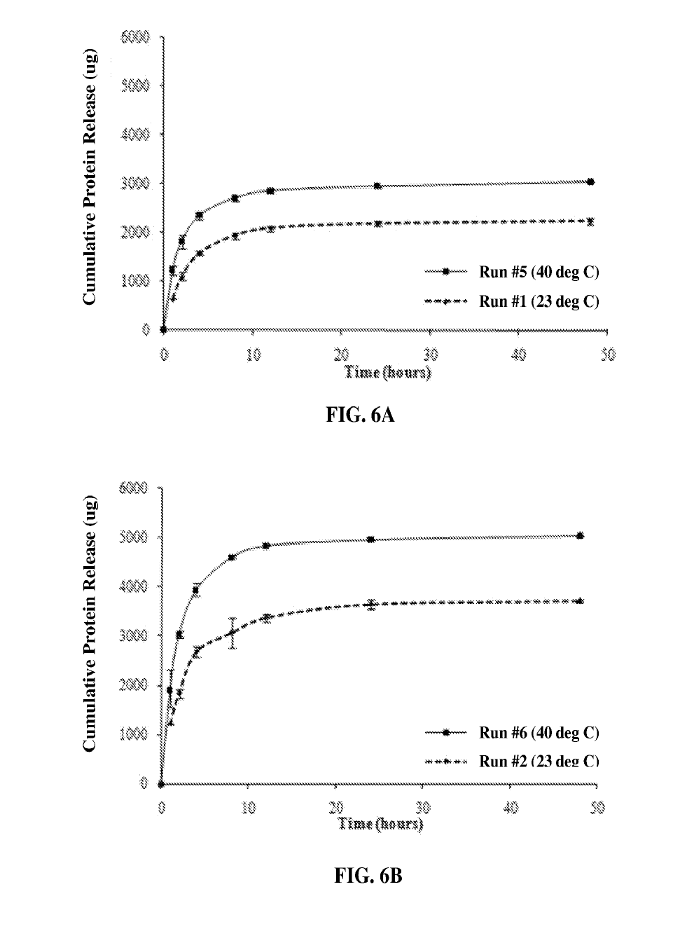

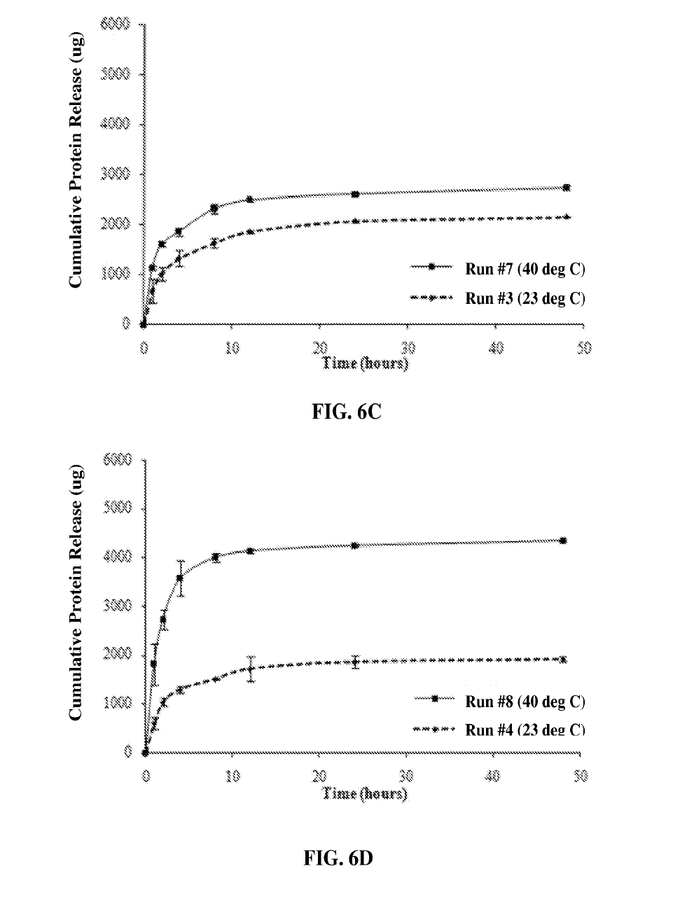

From the protein release data, all tests for the composite nanoparticle hydrogel network in Table 4 exhibited a triphasic protein release, where the triphasic protein release first includes an initial burst release (within the first hour), a second sustained burst release (from 1 to 8 hours), and followed by a third plateau release (from 8 up to 48 hours). In one embodiment, the initial burst release includes a rate between about 600-2000 μg/hr, the second sustained burst release includes a rate between about 10-400 μg/hr, and the third plateau release rate includes a rate between about 5-20 μg/hr. The rates can be adjusted by varying the factors indicated previously. Most of the protein was released within the first 8 hours, as shown in FIGS. 6A-D. In addition, all tests from Table 4 exhibited a thermoresponsive release behavior with hydrogels at 40° C. releasing a significantly higher amount of protein as compared to hydrogels at 23° C., over the same duration.

The protein release rates (R) may be calculated for the different tests at both temperatures (Table 4) in order to understand the factors that govern the protein release characteristics from the composite nanoparticle hydrogel network. Protein release rates (R) may be calculated using Equation (1):

$$R = \frac{{D}_{2} - {D}_{1}}{{t}_{2} - {t}_{1}} ; \qquad ( 1 )$$

where: R is the protein release rate between two time points (μg/hr); D₁ and D₂ are the amounts of protein (μg) released at times t₁ and t₂, respectively; and t₁ and t₂ are time points (hours) at which protein release was quantified.

Using the protein release rates (R), the factorial analysis is performed to evaluate the effect of individual factors on the protein release profiles. The release kinetics over these triphasic phase periods: 0 to 1 hour (initial burst release), 1 to 8 hours (sustained burst release), and 8 to 48 hours (plateau release). A half normal probability plot provides information on factors that are important and those that are not important, which provides insight into the relative importance of the individual factors on protein release.

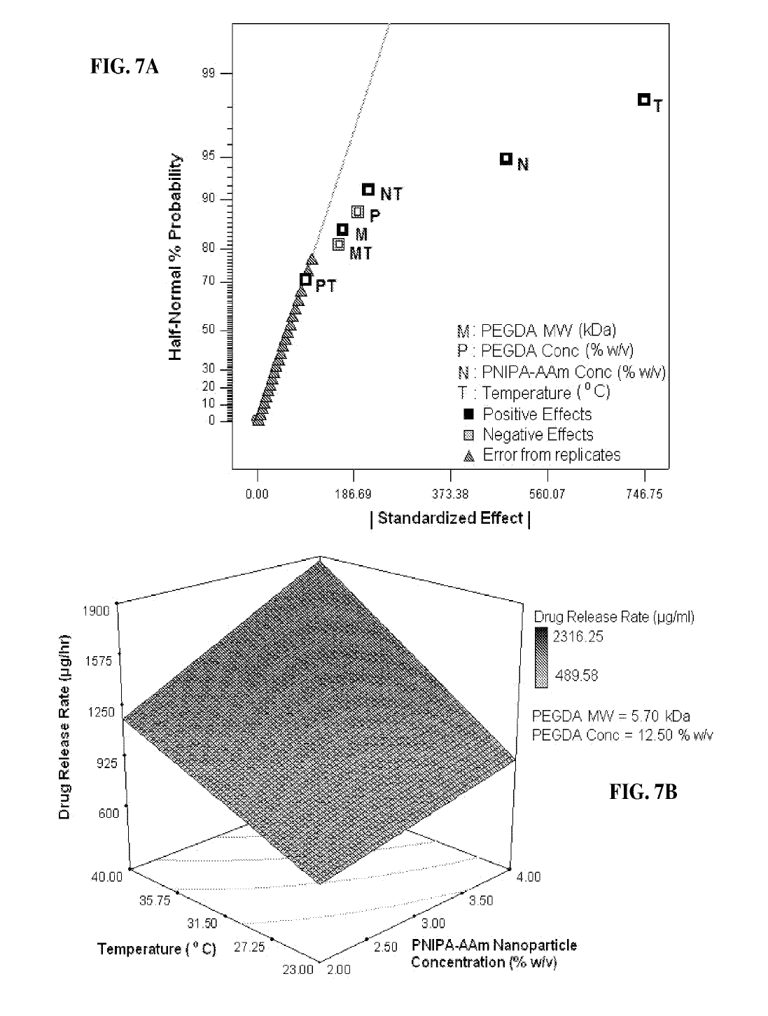

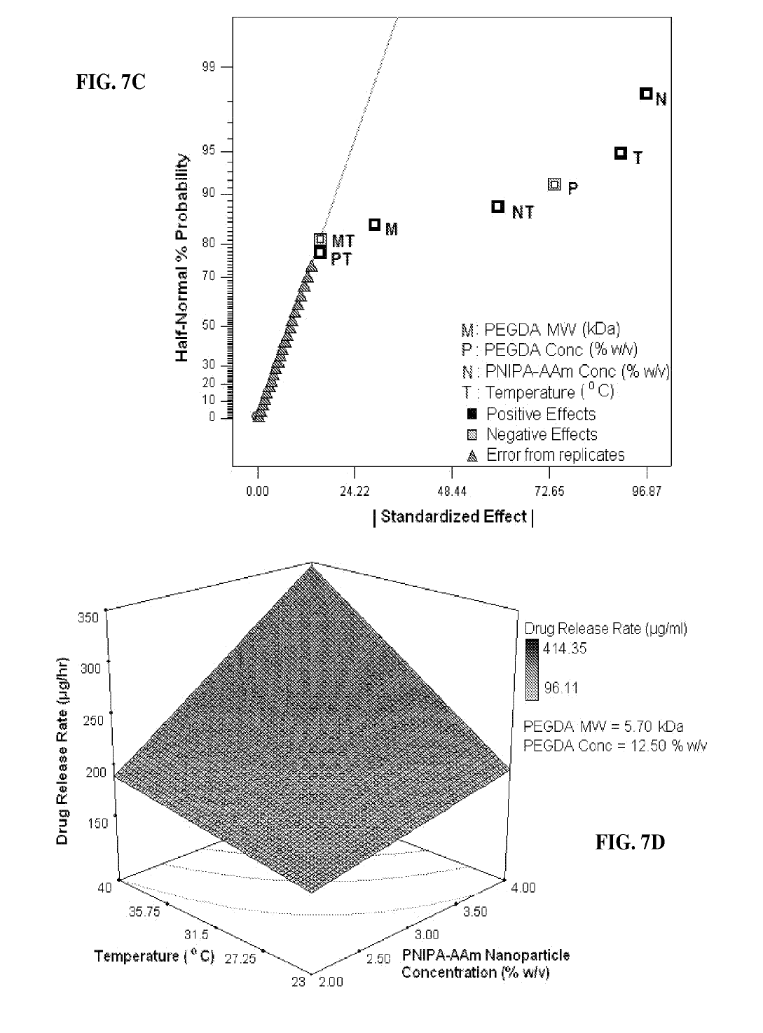

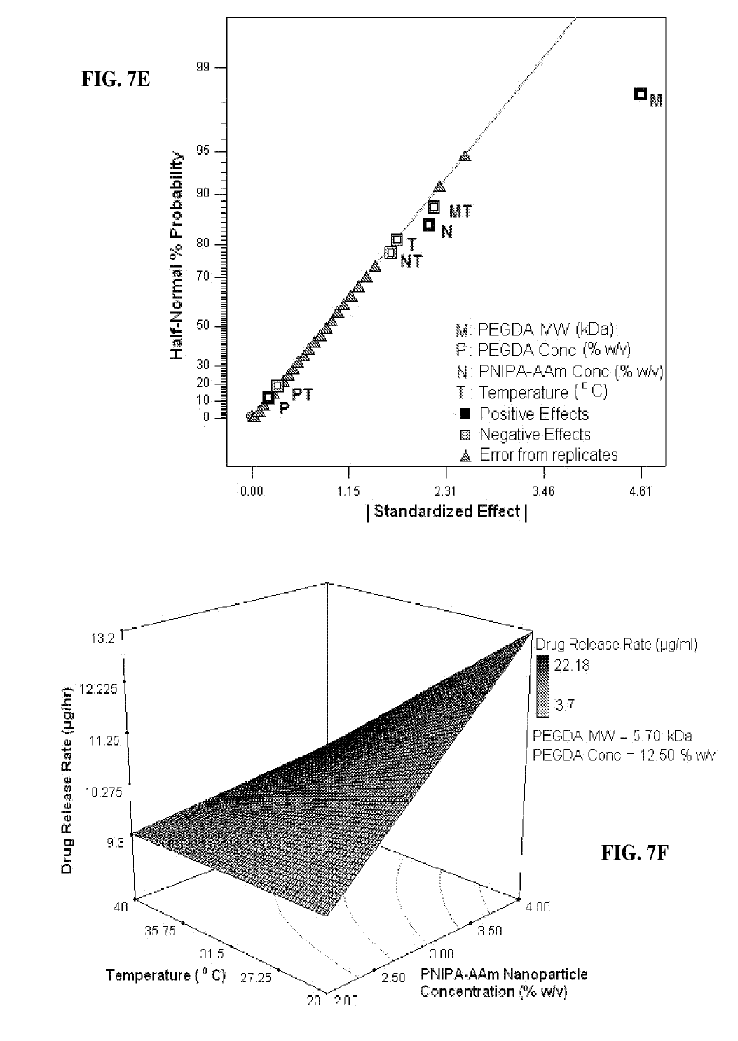

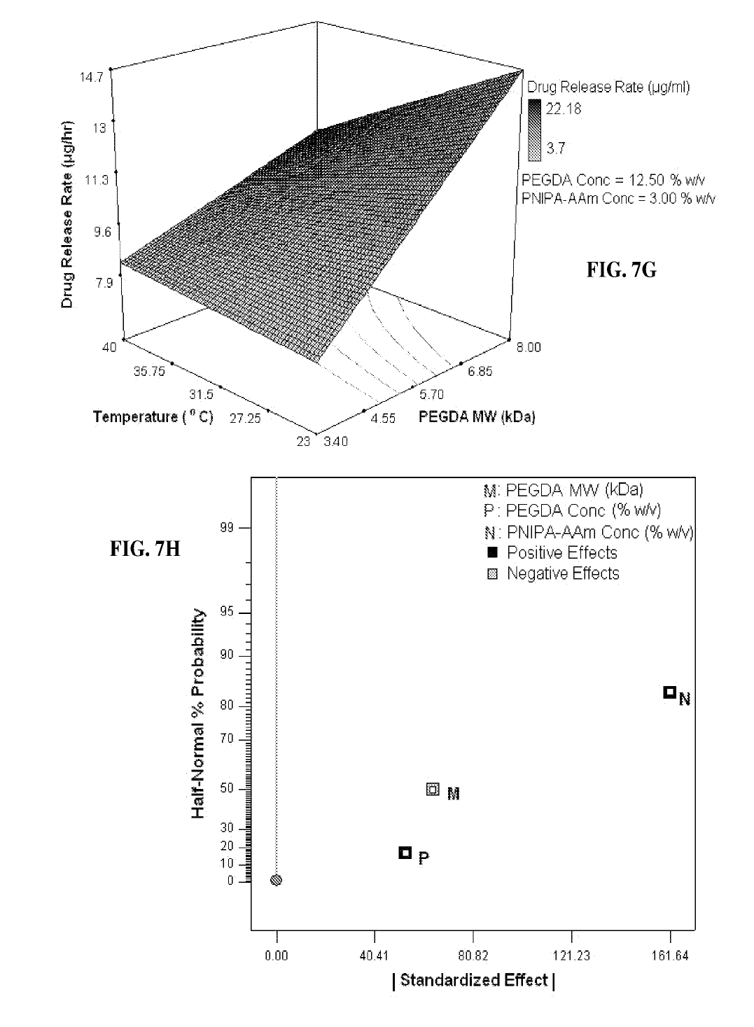

As shown in FIG. 7A, the absolute value of an effect on the X-axis and estimates of errors are represented as squares and triangles, respectively. The largest effects towards the right of the plot are real effects, while the effects close to the zero region are those that occur by chance and are categorized as errors. The effects are categorized as positive and negative effects. The positive effects represent a direct relationship of the design factors with the system outcomes, while the negative effects represent an inverse relationship. In the initial burst release (0-1 hour), the half normal probability plot shown in FIG. 7A indicates that the most important factors that control the release of the protein are the temperature, PNIPA-AAm concentration, and to a lesser extent a combination of the two. The PEGDA concentration (negative effect) and PEGDA molecular weight (positive effect) are less important to the rate of release. A response surface diagram is developed to show the relationship between PNIPA-AAm nanoparticle concentration and temperature on protein release rate. As shown in FIG. 7B, a response surface diagram with a PEGDA molecular weight of 5.7 kDa and a PEGDA concentration of 12.50% w/v, which lies close to the mid point of the range of both molecular weight and concentration. FIG. 4B shows that increasing the temperature have the single largest effect on burst release. The synergistic effect of temperature and PNIPA-AAm nanoparticle concentration also plays a significant role in enhancing the protein release rate.

PNIPA-AAm phase transition above LCST (i.e. a temperature above LCST) is the major factor in deciding the protein release rate. Most of the protein will be released when the PNIPA-AAm nanoparticles' structures collapse and expel the protein. The equation for protein release rate in the first initial burst release (0-1 hour) in terms of actual factors and combined effects of factors is obtained from factorial analysis. The predicted protein release can be calculated for any combination of individual factors within the range provided in Table 3 by using Equation (2):

R in the Initial Burst Release (μg/hr)=673.27+(163.78)M−(108.51)P−(161.21)N+(1.21)T−(4.06)MT+(2.21)PT+(12.75)NT; (2)

where: M=PEGDA molecular weight; P=PEGDA concentration; T=temperature; N=PNIPA-AAm nanoparticle concentration; MT is the combined effect of PEGDA MW and temperature; PT is the combined effect of PEGDA concentration and temperature; and NT is the combined effect of PNIPA-AAm nanoparticle concentration and temperature

In the second phase (sustained burst release, 1-8 hours), PNIPA-AAm nanoparticle concentration and temperature have positive effects, whereas PEGDA concentration has a significant negative impact on the protein release rate, as shown in FIG. 7C. A two factor interaction between PNIPA-AAm nanoparticle concentration and temperature also plays an important role. FIG. 7D shows the response surface plot for the dependence of the temperature and PNIPA-AAm nanoparticle concentration on the protein release rate. The equation for protein release rate from 1-8 hours, in terms of actual factors and combined effects of factors, is obtained from factorial analysis. The predicted protein release for the sustained burst release can be calculated for any combination of individual factors within the range provided in Table 3 using Equation (3):

R in the Second Sustained Burst Release (μg/hr)=464.34+(19.03)M−(26.37)P−(62.58)N−(7.55)T−(0.4)MT+(0.37)PT+(3.52)NT (3).

The effects of the factors on the protein release rate in the last plateau phase (sustained release, 8-48 hours) are different than in Phases I and II. Temperature and its combined effect with PNIPA-AAm nanoparticle concentration are not the important factors in controlling the protein release rate as shown in the half normal probability plot in FIG. 7E. The most dominant factor is the PEGDA molecular weight and the response surface diagram with temperature and PNIPA-AAm nanoparticle concentration is shown in FIG. 7F. The protein release rate at 40° C. is independent of the PNIPA-AAM concentration, while at 23° C., there is still a dependence on the PNIPA-AAm concentration. At 40° C., most of the protein has already been released in phase I and II and little protein is left for release in phase III, resulting in no dependence on the PNIPA-AAm concentration. On the other hand, at 23° C. a significant amount of protein is still present at the end of phase II, and the hydrogel networks with the higher PNIPA-AAm concentration have a higher release rate. As shown in FIG. 7G, the response surface diagram for when PEGDA MW and temperature are considered as variables. FIG. 7G shows a higher PEGDA MW results in a higher protein release rate, in particular at 23° C. The hydrogel networks with the higher PEGDA MW may allow more diffusion, resulting in a higher protein release rate in phase III.

A higher PEGDA concentration may result in a greater opportunity for cross-links to form, where the increased number of cross-links in the higher PEGDA MW might affect the network structure by forming a denser, closer knit network, thereby hindering the protein release in phases I and II of the release profile. Since protein release out of the composite nanoparticle hydrogel system occurs through diffusion, the lower porosity of the higher PEGDA MW results in larger retention of the protein in the earlier phases I and II, thereby resulting in a larger release in phase III (8-48 hours). The equation for the protein release rate from 8-48 hours, in terms of actual factors and combined effects of factors, is obtained from factorial analysis. The predicted protein release can be calculated for any combination of individual factors within the range provided in Table 3 using Equation (4):

R in the third phase (μg/hr)=−17.74+(2.75)M+(0.27)P+(4.12)N+(0.6)T−(0.06)MT−(0.0072)PT−(0.097)NT .(4)

Factorial analysis is performed on the photopolymerized composite nanoparticle hydrogel system to elucidate the relationship between the factors and the hydrogel thermoresponsive behavior, i.e. the higher protein release at 40° C. compared to 23° C. From FIGS. 6A-D, the composite nanoparticle hydrogel network included all four pairs of runs showing a significant thermoresponsive behavior. The release rate difference between 23° C. and 40° C. for all four pairs of runs is calculated and is shown in Table 4. The largest impact of the thermoresponsive behavior in release rates is in the first phase of the initial burst release of protein delivery.

Changing PNIPA-AAm nanoparticle concentration to the high level results in an increase in thermoresponsive behavior upon analyzing the factorial influence on the thermoresponsive behavior, as shown in FIGS. 7H and 7I. Higher concentrations of PNIPA-AAm nanoparticles elute larger amounts of protein when the temperature is raised above the LCST. As PNIPA-AAm nanoparticles are the thermoresponsive components of the system, the PNIPA-AAm particles have the greatest effect on the thermoresponsiveness. In addition, PEGDA molecular weight and PEGDA concentration affect the thermoresponsiveness by affecting the diffusion of the protein already expelled by nanoparticles and entrapping the proteins in the hydrogel network.

The swelling ratios for the composite nanoparticle hydrogel networks from different runs are determined to understand how the factors such as PEGDA molecular weight and concentration as well as PNIPA-AAm nanoparticle concentration affected the hydrogel structure. After photopolymerization, the composite nanoparticle hydrogel networks (n=4) are allowed to swell with a PBS solution at room temperature for 4 days. The swollen PBS composite nanoparticle hydrogel networks were then dried with moistened filter paper and weighed to get the swollen weight (W_{S}) of the composite nanoparticle hydrogel networks. The dry weight (W_{D}) of the composite nanoparticle hydrogel networks are measured after the drying of the composite nanoparticle hydrogel networks. The swelling ratio (S.R.) of the hydrogels is calculated using Equation (5):

$$S . R . = \frac{{W}_{S} - {W}_{D}}{{W}_{D}} . \qquad ( 5 )$$

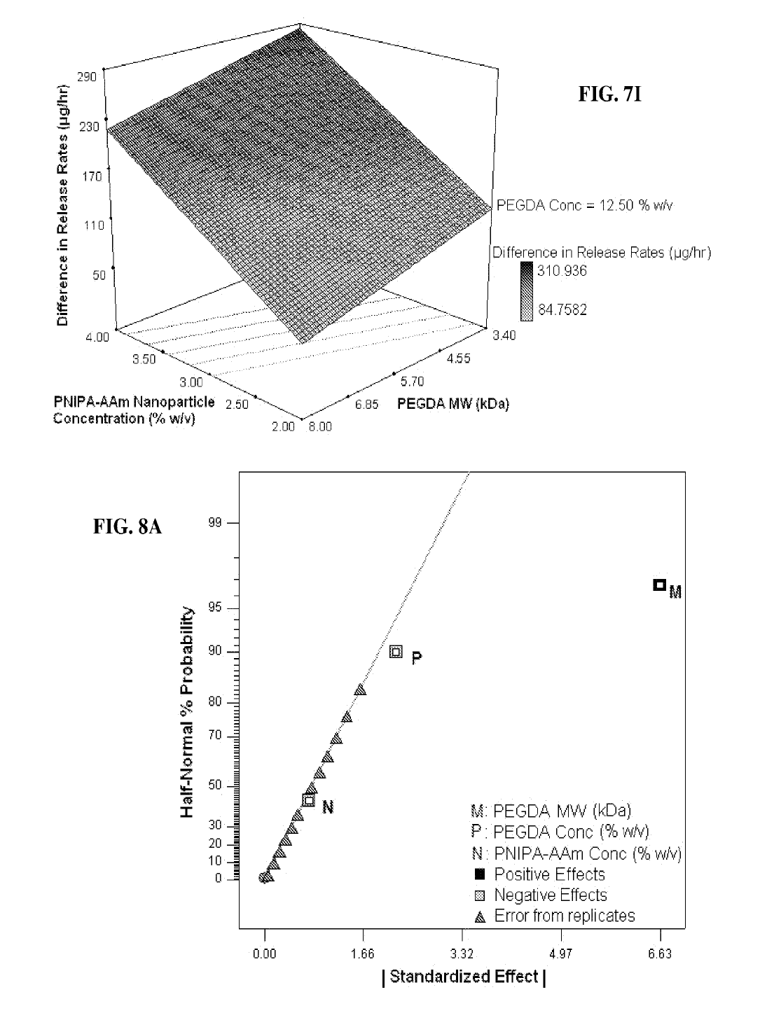

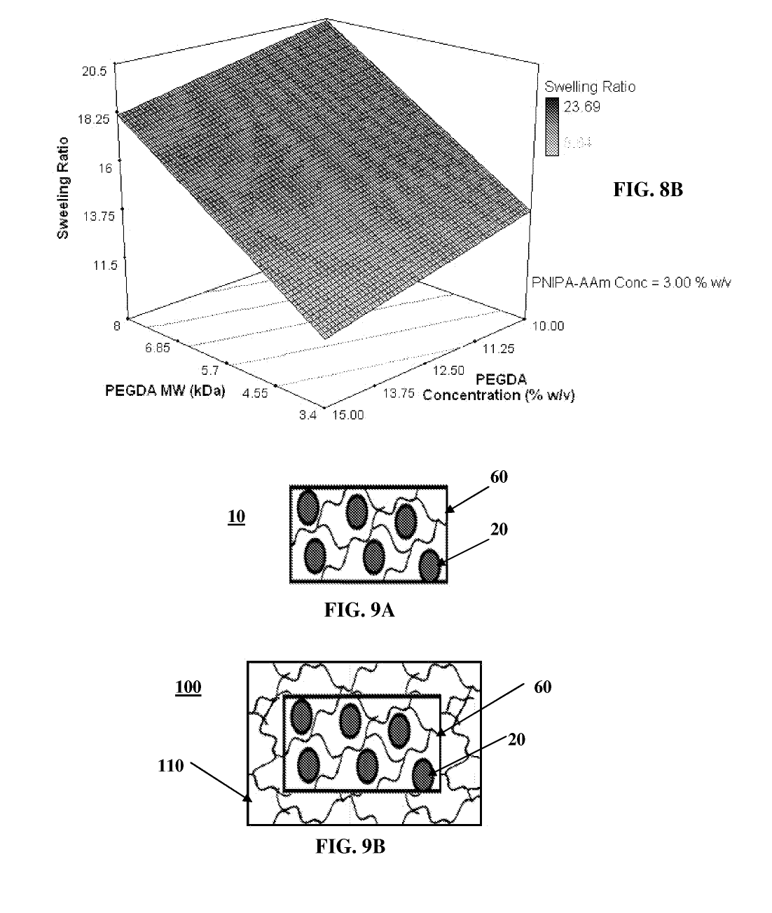

To evaluate the effects of the factors, excluding temperature, on the swelling ratio, the swollen weights (W_{S}) and dry weights (W_{D}) of the composite nanoparticle hydrogel networks (n=4) are measured. The swelling ratios of the composite nanoparticle hydrogel networks are then calculated using Equation (5). The factorial analysis on the composite nanoparticle hydrogel networks swelling ratio revealed that increasing the PEGDA MW from 3.4 kDa to 8 kDa was the most important factor in increasing the swelling ratio, as shown in FIGS. 8A and 8B. PEGDA concentration and PNIPA-AAm nanoparticle concentration are found to have a mild, negative effect on the swelling ratio.

The composite nanoparticle hydrogel network swelling ratios shows the lower MW cross-linker has shorter chains than the higher MW cross-linker, and thus forms a tighter, more compact network due to the larger number of cross-links. Therefore, a lower MW cross-linker will not allow the composite nanoparticle hydrogel networks to swell sufficiently (compared to the higher MW), and hence, diffusion of water into and protein out of the composite nanoparticle hydrogel network would be limited. Increasing the PEGDA MW has a positive effect on the protein release, which is more pronounced in the third plateau release of the protein release, where a significant portion of the protein is already released and the remaining protein release is controlled by the diffusion mechanism, which is shown in FIG. 7G, as the higher PEGDA MW has a higher release rate compared to the lower PEGDA MW.

In one embodiment, midpoint analysis may be performed to confirm the linear dependence of the dependent variables on the independent variables. Midpoint analysis includes the approximate mid-levels of three factors other than temperature, which are chosen and composite nanoparticle hydrogel networks were prepared (n=4) for midpoint analysis. For example, the precursor solution is prepared by selecting a PEGDA molecular weight of 6 kDa and a concentration of 12.5% (w/v) and a PNIPA-AAm nanoparticle concentration of 3% (w/v). The composite nanoparticle hydrogel networks are formed by adding 200 μl of the precursor solution to a 48-well plate and exposed to UV light at about 10 mW/cm². To evaluate the effect of the factors on protein release, composite nanoparticle hydrogel networks (n=4) are incubated at room temperature (23° C.) and above LCST (40° C.) in 24-well plates with 1 ml of PBS solution. Protein release studies are performed as described previously.

As shown in FIG. 9B, the double layer (DL) composite nanoparticle hydrogel networks 100 may be prepared to tailor the protein release rate and minimize the first initial burst release. In order to form the double layer composite nanoparticle hydrogel networks, single layer composite nanoparticle hydrogel networks are first prepared, as previously described, by selecting midpoint values of the factors as mentioned in the midpoint analysis. As shown in FIG. 9A, the single layer composite nanoparticle hydrogel networks 10 (n=4), immediately after formation, are immersed in the solution containing PEGDA and the photoinitiator in a 24-well plate. The solution containing the single layer hydrogel plus the PEGDA and photoinitiator is then exposed to UV light and photopolymerized to form an additional cross-linked layer of PEGDA 110 around the single layer composite nanoparticle hydrogel network 10. Protein release studies were performed by incubating double layer composite nanoparticle hydrogel networks at 23° C. and 40° C. to compare with the protein released from single layer composite nanoparticle hydrogel networks (prepared for midpoint analysis). Alternatively, the cross-linked layer of PEGDA 110 may embed additional PNIPA-AAm nanoparticles with additional or different bioactive molecules.

Midpoint analysis is conducted through the protein release to evaluate the curvilinear effect of dependent factors on the independent factors. The scheme of the double layer composite nanoparticle hydrogel networks and the cumulative protein release from single layer (SL) and double layer (DL) composite nanoparticle hydrogel networks at 23° C. and 40° C. are shown in FIGS. 9A and 9B. Consequently, the drug released from PNIPA-AAm nanoparticles slowly diffused first through the PEGDA network and then through the outer layer of PEGDA. Thus the double layer composite nanoparticle hydrogel system 100 can be used to release the drug over a longer period of time.

The protein release profiles of both types of composite nanoparticle hydrogel network exhibited a thermoresponsive release behavior with composite nanoparticle hydrogel networks at 40° C. releasing a significantly higher amount of protein, compared to composite nanoparticle hydrogel networks at 23° C., over the same time duration. The double layer composite nanoparticle hydrogel networks release a significantly smaller amount of protein in a sustained manner compared to the single layer composite nanoparticle hydrogel networks.

The protein release studies on composite nanoparticle hydrogel networks at the midpoint level of the two level factorial design (midpoint analysis) generated similar protein release profiles to the composite nanoparticle hydrogel networks of the four run pairs, as shown in FIG. 9C. The protein release profiles exhibited the triphasic protein release and thermoresponsive release behavior. In addition, the effects of individual and combined factors on protein release profiles during each phase of release and swelling ratio were similar to the studies shown in FIGS. 7B, 7D, 7G, and 7B. These protein release profiles confirm the linear dependence of dependent variables on the independent variables. The results from DL composite nanoparticle hydrogel networks, with an outer layer of PEGDA that was protein-free surrounding and the inner layer of PEGDA that contained the protein-loaded PNIPA-AAm nanoparticles, indicate that the protein released from PNIPA-AAm nanoparticles slowly diffused first through the PEGDA network and then through the outer layer of PEGDA. Thus, the double layer composite nanoparticle hydrogel networks could be used to provide a more sustained and controlled drug/protein release in response to changes in temperature compared to single layer hydrogels.

Composite nanoparticle hydrogel networks can be photopolymerizable in situ and can release drugs in response to changes in local temperature for drug/protein delivery applications. The system comprises the drug- or protein-loaded PNIPA-AAm nanoparticles, photoinitiators (Irgacure 2959), and PEGDA photo cross-linkers. The composite nanoparticle hydrogel system can form a hydrogel network at any shape or form under the presence of UV light within a short time (less than 5 minutes). In addition, the temperature within the hydrogel raised by a short UV exposure is below the body temperature (<32° C.), so there is no protein denatured issue in this system. The percentage of protein denaturation may be maintained at or about 0-5% of protein released from the PNIPA-AAm nanoparticles, which is suitable for delivering stable proteins in their native monomeric form. Percentage of denatured proteins released from the hydrogel network may be determined by gel electrophoresis and an analysis on the gel samples for the percentage of denatured proteins.

When the local temperature is increased to or above the LCST (39-40° C.), the PNIPA-AAm nanoparticles undergo a reversible phase transition, collapse, and expel the drugs into the surrounding tissue, so the composite nanoparticle hydrogel system can be used for on-off drug delivery mechanism. The factorial analysis evaluates the effects of four factors (PEGDA MW and concentration, PNIPA-AAm nanoparticle concentration, and temperature) on protein release, thermoresponsiveness, and swelling ratio of the hydrogels. For protein release, in the initial burst (phase I) and sustained burst region (phase II), higher PNIPA-AAm nanoparticle concentration and higher temperature were shown to result in an increase in protein release, while PEGDA MW governed protein release in the plateau region (phase III). PNIPA-AAm nanoparticle concentration was the major factor controlling the degree of thermoresponsiveness of the hydrogel systems with systems consisting of larger PNIPA-AAm concentration having higher release rates. On the other hand, PEGDA MW was found to be the most important factor for swelling ratio with higher MW PEGDA having higher swelling ratios. Furthermore, a sustained release of drug or protein can be achieved by adding another layer of PEGDA on top of the composite hydrogel. The composite hydrogel system can be tailored to obtain desired characteristics such as drug release profiles and swelling.

Photocrosslinkable hydrogels for protein release does not cause significant denaturation of loaded proteins. Preliminary size exclusion chromatography studies indicated that the released BSA was almost entirely in its native monomeric form as indicated by Leach J B, Schmidt C E. “Characterization of protein release from photocrosslinkable hyaluronic acid-polyethylene glycol hydrogel tissue engineering scaffolds”, Biomaterials 2005 January; 26(2):125-135, herein incorporated by reference.

### EXAMPLE 1

### Cytocompatibility Studies of an In Situ Composite Nanoparticle Hydrogel System Using Human Aortic Smooth Muscle Cells

The in situ photopolymerized thermoresponsive composite nanoparticle hydrogel system may aid in the prevention of restenosis after angioplasty. Coronary balloon angioplasty involves clearing the blocked artery by inflating a balloon and compressing the plaque against the arterial wall, commonly resulting in damage to the endothelial layer. Restenosis, the renarrowing of the treated artery, is caused by a major loss of the endothelial cell population (a natural vascular barrier), resultant smooth muscle cell (SMC) migration, and subsequent SMC proliferation at the injured arterial wall site. The composite nanoparticle hydrogel system comprises poly (N-isopropylacrylamide) (PNIPA) thermoresponsive nanoparticles, photo cross-linker poly(ethylene glycol) diacrylate (PEGDA), and a UV photoinitiator 2-hydroxy-1-[4-(2-hydroxyethoxy)phenyl]-2-methyl-1-propanone (Irgacure 2959), which is photopolymerized at the injured wall with exposure to UV light following angioplasty for local drug delivery.

The composite nanoparticle hydrogel system provides both local and stimuli-responsive drug delivery capable of releasing a drug in response to temperature changes. The drug would be selected based on its ability to prevent further human aortic smooth muscle cell (HASMC) migration and proliferation, major causes of restenosis. In addition to releasing the drug in a temperature-responsive manner, the hydrogel network also acts as a protective barrier against the recruitment of blood cells such as platelets and leukocytes, which are major causes of thrombosis and inflammation at the damaged arterial wall. The composite nanoparticle hydrogel system quickly photopolymerizes at the injured arterial wall after angioplasty upon exposure to UV light, photopolymerization allows rapid conversion of a liquid monomer or macromer solution into a gel in situ. The photopolymerized composite nanoparticle hydrogel network include spatial and temporal control of reaction kinetics, fast-curing rates to provide rapid polymerization, and effective control over cross-linking density, thereby governing the release rate. These advantages make photopolymerized hydrogels extremely desirable as systems for smart local drug delivery.

The components needed to form photopolymerized composite nanoparticle hydrogel network are the photo cross-linker, the photoinitiator, and UV irradiation in the precursor solution, as described previously. Poly(ethylene glycol) (PEG) functionalized with diacrylate group (PEGDA) cross-links quickly in the presence of UV light and a photoinitiator to form hydrogels. Additionally, PEGDA is biocompatible and nontoxic as it is a derivative of PEG. When the photoinitiator molecules are exposed to specific wavelengths of visible or UV light, the photoinitiator molecules dissociate into free radicals that initiates the polymerization reaction. The UV photoinitiator Irgacure 2959 was selected as the most cytocompatible UV photoinitiator compared to other photoinitiators for different cell types. The effect of Irgacure 2959 on different cell types displayed different sensitivities to the same concentration of this photoinitiator, so the sensitivity of HASMCs specifically to Irgacure 2959 must be determined.

The use of UV light and photoinitiator molecules may affect the compatibility of these systems in biomedical applications. The cells can undergo cellular damage during photopolymerization as a result of exposure to photoinitiator molecules, reactive macromers, and free radicals. For composite nanoparticle hydrogel network system, inhibiting HASMC migration and proliferation is necessary to prevent restenosis. The photoinitiating system must not have a deleterious effect on the existing HASMC population. Thus, the biocompatibility of the composite nanoparticle hydrogel system on HASMCs must be evaluated and the cytotoxicity of its components must be minimized. Therefore, the cytotoxic effects of the composite nanoparticle hydrogel system components on the HASMCs must be evaluated. First, HASMCs were exposed to different photoinitiator concentrations with or without UV light exposure for various periods. Cell survival was then determined by MTS assays. Ascorbic acid, an antioxidant, was also tested for its efficiency in reducing the cytotoxicity of free radicals. In addition, the effect of antioxidant addition on the gelation time of the composite nanoparticle hydrogel network was performed and evaluated. Finally, the media was incubated with the composite nanoparticle hydrogel network for 8 h, and cell survival was determined after HASMCs were incubated with this media for 3 days.

Methods and Results

Chemicals, if not specified, were purchased from Sigma-Aldrich (St. Louis, Mo.), including N-isopropylacrylamide (NIPA), N,N′-methylenebisacrylamide (BIS), potassium persulfate, and sodium dodecyl sulfate. Statistical analysis of the results was performed using ANOVA and t tests with p<0.05 (StatView 5.0 software, SAS Institute). Post hoc comparisons were made using the Fisher's least significant differences. For each, four samples were tested (n=4), and all the results are given as mean±SD.

Human aortic smooth muscle cells (HASMC) were cultured in complete medium consisting of Dulbecco's modified Eagle medium supplemented with 10% fetal bovine serum and 1% penicillin-streptomycin (Invitrogen, Carlsbad, Calif.). Upon 80-90% confluency, the cells were passaged or used for experiments. For all experiments, the cells were seeded in 24-well plates (Corning, Corning, N.Y.) at a density of 7000 cells per well. Following seeding, the cells were incubated at 378C and 5% CO₂ in a humid environment for 2 days to allow cellular attachment and growth. After 2 days, the HASMCs were exposed to varying concentrations of the photoinitiator Irgacure 2959 (Ciba Specialty Chemicals), and/or UV exposure.

PNIPA nanoparticles were first prepared using the methods described above. Photopolymerized hydrogels were then produced using the method outlined above. Briefly, PNIPA nanoparticles (20% wt of PEGDA) were added to a solution containing PEGDA (3400 MW) with a final PEGDA concentration of 100 mg/mL. The UV photoinitiator, Irgacure 2959, was then added at a final concentration of 0.015% (w/v). Two hundred microliters of this solution was added to a 48-well plate and exposed to long wave, 365-nm UV light at about 10 mW/cm² for 5 min to form the composite hydrogels.

To evaluate the effects of UV exposure on cell survival, cells were seeded and cultured as described earlier. The cells were then exposed to varying durations (1, 3, and 5 min) of long wave, 365-nm UV light (Model B-100AP/R, UVP) at about 10 mW/cm². These durations of UV exposure were chosen, because they are sufficient to photopolymerize the hydrogels. Cells not exposed to UV light served as the control and were used to determine the relative cell survival rate. Following exposure, the cells were incubated for another 3 days before quantifying the cell survival. The cell survival for controls was determined to be 1±0.09. The effects of varying durations of UV exposure on the HASMC survival are shown in FIG. 7. Exposure of HASMCs to 1, 3, and 5 min did not show any statistically significant decrease in cell survival, and the relative cell survival rates were 0.96±0.05, 0.97±0.04, and 1.07±0.02, respectively.

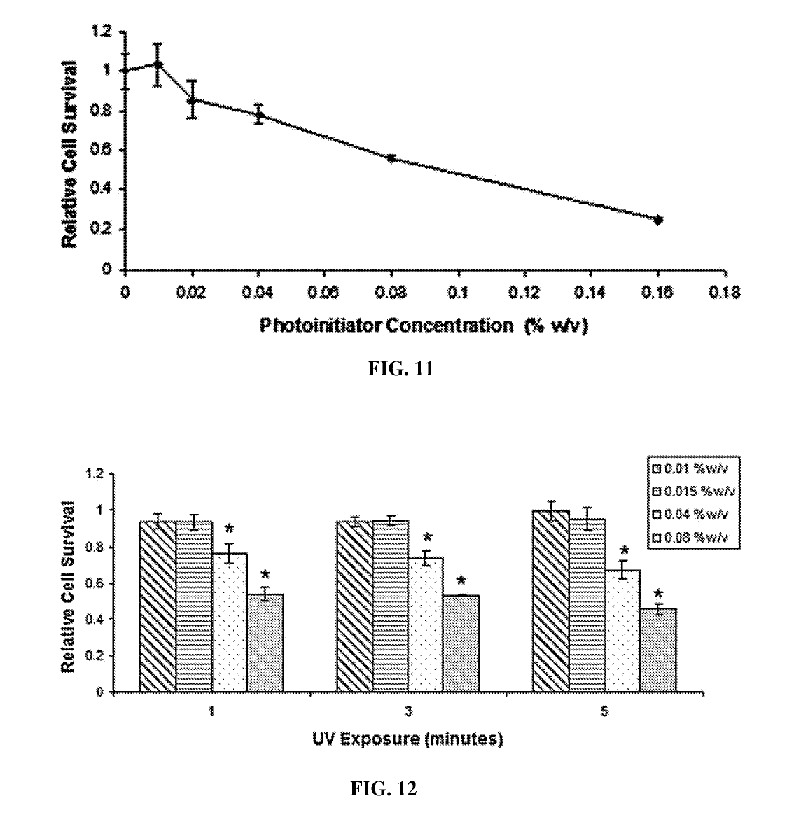

To evaluate the cytotoxic effects of photoinitiator concentrations on HASMCs, the cells were seeded and cultured as described earlier. Irgacure 2959 was directly dissolved in complete media to obtain final concentrations of 0.01%, 0.02%, 0.04%, 0.08%, and 0.16% (w/v). These photoinitiator concentrations were well within the range required for photopolymerization in a short period of time. The photoinitiator solutions were carefully protected from exposure to light to preserve their activity. These solutions were then sterilized using 0.2-μm syringe filters before they were added to the HASMCs. The control wells consisted of cells incubated with photoinitiator-free complete media. After the addition of the photoinitiator, the cells were incubated for 3 days, and then cell survival was determined using MTS assays. The cell survival for the controls was determined to be 1±0.09. The cytotoxic effects of the varying photoinitiator F2 solutions are shown in FIG. 11. Irgacure 2959 did not show a significant decrease in cell survival when HASMCs were exposed to 0.01% (w/v) photoinitiator solution, and relative cell survival was determined to be 1.03±0.10 (n 5 4). Upon increasing the photoinitiator concentrations above 0.02% (w/v), a statistically significant decrease was noticed in the relative cell survival. Varying the photoinitiator concentration from 0.02% (w/v) to 0.16% (w/v) was found to progressively decrease the relative cell survival from 0.85±0.09 to 0.25±0.01, respectively.

The cellular damage due to the combined effects of photoinitiator and UV exposure was evaluated. Briefly, the HASMCs were seeded onto 24-well plates and allowed to grow for 2 days. Irgacure 2959 solutions with final concentrations of 0.01%, 0.015%, 0.04%, and 0.08% (w/v) in complete media were prepared. After adding these solutions, cells were incubated for 30 min to allow for the mixing of the photoinitiator. The well plates were then exposed to 1, 3, and 5 min of UV light. Wells containing cells not exposed to either UV light or photoinitiator solution served as controls for this experiment. Cell samples were incubated for 3 days before analyzing the cell survival. As shown in FIG. 12, the relative cell survival was calculated using F3 the control wells, which were not exposed to the photoinitiator solution or UV light. The cell survival for the controls was determined to be 1±0.09. For 0.01% and 0.015% (w/v) of photoinitiator concentrations, the HASMCs did not show any statistically significant decrease in relative cell survival for 1, 3, and 5-min exposure (n=4). At 0.04% (w/v) concentration of Irgacure 2959 in complete media, a significant decrease was observed at 1, 3, and 5 min of UV exposure, with relative cell survival values at 0.76±0.05, 0.74±0.04, and 0.67±0.05 of the control samples, respectively. Finally, the relative cell survival rates were significantly reduced at 0.08% (w/v) photoinitiator concentration, ranging between 0.54±0.04 and 0.45±0.03 for the three durations of UV exposure.

The efficiency of the antioxidant, ascorbic acid, was evaluated in scavenging the free radicals in an effort to increase cell survival. HASMCs were exposed to 0.15% (w/v) solution of Irgacure 2959 in complete media supplemented with varying concentrations of ascorbic acid (0-200 mg/L). After incubating for 30 min, the cells were exposed to 5 min of UV light, and cell survival was quantified after 3 days. To evaluate the effect of added antioxidants on the gelation time, the gelation times (i.e., the time required for the materials to form a gel) were determined using three methods on 96-well plates. For this, 50 mg/L of ascorbic acid was added to the hydrogel precursor solution, containing 0.15% (w/v) Irgacure 2959. In the first method, the gels were exposed to the UV light, and viscosities of the gel solution were observed. The end point was demonstrated when the gel was picked up with the pippet tip. In the second method, a stir bar was placed in a well containing the hydrogel solution, and the gelation time was defined as a time required for the stir bar to stop stirring. In the last method, UV-vis spectrophotometer was used to monitor for the change in the intensity of the gelation solution from 340 to 1020 nm wavelength to choose the optimal wavelength. The highest change in the intensity was observed at 610 nm. Then, the solution was monitored at that this specific wavelength AQ3 (610 nm) over a time course. The result was graphed as function of time, and the gelation time was defined as the time that had the highest changes in the absorbance intensities.

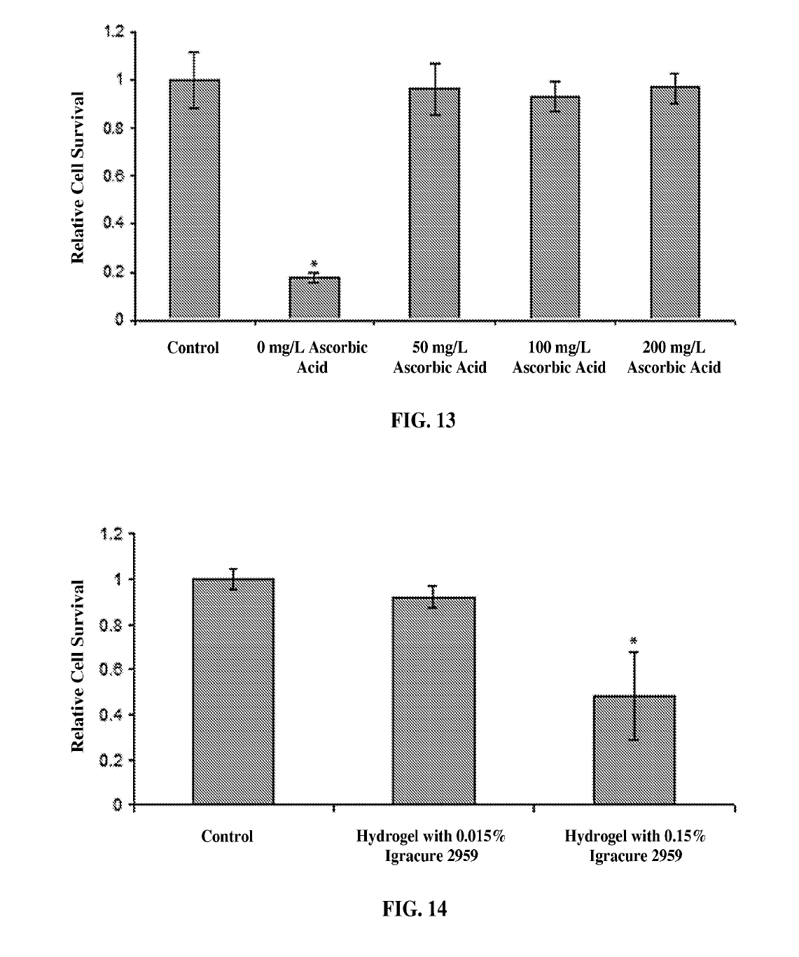

Even at low concentrations (50 mg/L), ascorbic acid was able to significantly improve the relative cell survival rates compared to F4 samples without ascorbic acid, as shown in FIG. 13. In addition, the effect of antioxidant addition on the gelation time of the hydrogel was investigated. Hydrogels with 50 mg/L of ascorbic acid required more time to form when compared with hydrogels with no antioxidants, with all other conditions remaining constant is shown in Table 6, where ^{a} is the significant difference compared to samples without ascorbic acid, p<0.05.

TABLE 6
Effects of Antioxidants (e.g., Ascorbic Acid) on the Gelation Time

| | Gelation Time (min) | | |
|---|---|---|---|
| Samples | Observation | Stirring | Spectrophotometer |
| Without | 3.44 ± 0.230 | 3.59 ± 0.290 | 4.00 ± 0.009 |
| Ascorbic Acid | | | |
| With | 4.05 ± 0.100^{a} | 4.20 ± 0.060^{a} | 4.42 ± 0.016^{a} |
| Ascorbic Acid | | | |

The cytotoxic effects of the photopolymerized composite nanoparticle hydrogel network on the HASMC population were performed to evaluate how the whole composite hydrogel system affected cell survival. Here, hydrogels (n=4) were photopolymerized (as described earlier) with 0.015% and 0.15% (w/v) Irgacure 2959 concentrations. HASMC media were then incubated with the hydrogels for 8 h to allow the leaching of all potential cytotoxic components from the hydrogels into the cell media. After 8 h, this medium was added to the HASMCs, which had been grown for 2 days. For the controls, fresh media (not incubated with hydrogels) were added to the cells. After 3 days of incubation, the cell survival was evaluated using MTS assays.

After the experiments, the cell survival was quantified using the MTS assay (CellTiter 961 Aqueous One Solution Cell Proliferation Assay, Promega) following the manufacturer's instructions. The cells were incubated with the MTS reagent for 4 h, after which 200 μL of the solution was transferred to 96-well plates, and absorbance was read at 490 nm using a microplate reader (VMax, Molecular Devices). Relative cell survival was obtained by dividing the absorbance reading of a cell sample by the mean absorbance value of the control.

The cytotoxic effects of the photopolymerized composite nanoparticle hydrogel networks were evaluated. Hydrogels were photopolymerized with 0.015% and 0.15% (w/v) Irgacure 2959 concentrations and then incubated with HASMC media for 8 h. This media was then added to the seeded HASMCs, and the relative cell survival rates for cells incubated with hydrogel media for 3 days are shown in FIG. 14. Controls exhibited a relative cell survival F5 rate of 1±0.04. For hydrogels with 0.015% (w/v) Irgacure 2959, relative cell survival of HASMCs did not show any significant decrease (0.93±0.04). For HASMCs incubated with media from hydrogels photopolymerized with 0.15% (w/v) Irgacure 2959, relative cell survival was significantly decreased compared to the controls (0.48±0.19 of controls).

Discussion

Photopolymerizable hydrogel systems have been used in several applications including drug delivery and tissue engineering. The ability to rapidly form a hydrogel in situ using photopolymerization makes this system highly desirable for biomedical applications. The thermoresponsive composite nanoparticle hydrogel system may aid in preventing restenosis. Following angioplasty, the composite nanoparticle hydrogel system would be photopolymerized at the site of the injured arterial wall to release drugs that inhibit restenosis. The drug-delivery system components should not cause additional damage to the surrounding cells and tissues. Hence, the cytocompatibility of the components of the photoinitiating system, including UV light and the photoinitiator was evaluated. HASMCs, normally present at an injured site, were chosen for the evaluation of the cytotoxic effects of the system components.

Short-time exposure to UV light did not cause significant cytotoxicity, as shown in FIG. 10. By evaluating the cytotoxicity of varying times of UV exposure, it showed that UV light did not cause any significant decrease in HASMC survival (1.07±0.02) after 5 min of exposure. The effects of UV exposure on six different cell types, including human fetal osteoblasts, bovine chondrocytes, rabbit corneal epithelial cells, human mesenchymal stem cells, goat mesenchymal stem cells, and human embryonic germ cells may find that short UV exposure (5 min) do not alter cell survival significantly. These results, combined with the data presented, confirm that while its cytotoxic effects may vary slightly among cell types, UV exposure does not significantly contribute to cell death at conditions (short-time exposure) required for photopolymerization.

The effects of different photoinitiator concentrations on the survival of HASMCs were evaluated, as shown in FIG. 11. The cytocompatibility of Irgacure 2959 was evaluated, as HASMCs would be exposed to the initiator molecules before polymerization. Additionally, some undissociated initiator molecules could possibly harm the cells after polymerization. The cytotoxicity of different, commercially available, visible light and UV photoinitiators. cytocompatibility of four UV photoinitiators on NIH/3T3 fibroblast cells and found Irgacure 2959 to be the most cytocompatible UV photoinitiator that does not affect fibroblast survival at concentrations 0.05% (w/w). These studies also observed positive results on chondrocytes. Irgacure 2959 is the most cytocompatible amongst these six cell lines with different cell types reacting differently to the same concentration of a single photoinitiator. On the basis of the results from these studies, Irgacure 2959 was selected as a photoinitiator for the composite nanoparticle hydrogel system, and thus, the cytocompatibility of Irgacure 2959 specific to HASMCs was evaluated. HASMCs showed a higher sensitivity to Irgacure 2959 compared to different cell types studied by other groups. Below 0.02% (w/v) concentration, there was no significant decrease in cell survival. At concentrations greater than 0.02% (w/v), the cytotoxicity of Irgacure 2959 increased with increasing photoinitiator concentrations and significantly affected the cell survival. Low photoinitiator concentrations (0.015%) can photopolymerize the composite nanoparticle hydrogel networks within short periods of UV exposure (5 min). The HASMCs were exposed to Irgacure 2959 for 3 days, whereas, in vivo, the cells would be exposed to the photoinitiators for a shorter period, i.e. less than 1 hour.

The cellular damage caused by photopolymerization was also evaluated by the combined effects of photoinitiator molecules and UV light, as shown in FIG. 12. As initiator molecules dissociate into free radicals upon exposure to UV light, and free radicals can damage cellular membranes. At lower photoinitiator concentrations (0.01% and 0.015% w/v) and UV-exposure times (1, 3, and 5 min), the cell survival rates were not statistically different from the controls. At 0.04% and 0.08% (w/v) photoinitiator concentrations, a significant decrease was noted in cell survival rates for all durations of UV exposure. For high-photoinitiator concentrations, the increased cell death might be attributed to the free radicals released during photopolymerization. Furthermore, the cells may also be exposed to a toxic environment consisting of initiator by products and undissociated initiator molecules in addition to generated free radicals following polymerization.

To reduce the cytotoxic effects, the free radicals released during the photopolymerization process may be scavenged. The presence of antioxidant ascorbic acid in the culture media might reduce the sensitivity of the cells to the toxic effects of Irgacure 2959. The ability of ascorbic acid to scavenge free radicals and to reduce photoinitiator toxicity was evaluated and was found that ascorbic acid, even at low concentrations (50 mg/L), significantly increased the cell survival. Different cell types might respond differently to a single photoinitiator due to variations in their expression of antioxidant enzymes, receptors for antioxidant enzymes, and addition of antioxidants to their culture media. The results confirm that the cell survival rates may be significantly altered by adding an antioxidant, as shown in FIG. 13. The free radicals are critical to the polymerization process, so the presence of ascorbic acid altering the gelation time of the hydrogel was determined. Upon addition of 50 mg/L concentration of antioxidant ascorbic acid, the time required for gelation was increased compared to the gelation time in the absence of an antioxidant. Although the addition of ascorbic acid significantly improved cell survival, its increased gelation time may result in increased photoinitiator and UV exposure, making alternative antioxidant attractive for the composite nanoparticle hydrogel system. Alternative antioxidants may include glutathione, melatonin, vitamin A, C, D, E, non-phenolic antioxidants, as well as enzymes such as catalase, superoxide dismutase and various peroxidases.

Finally, the cytotoxic effects of the photopolymerized composite hydrogels as a whole were evaluated. During photopolymerization, the reactive macromers react with the free radicals, thereby possibly reducing the harmful effects of the radicals on the cells. Thus, there was a potential cytotoxic effect of all components in the drug-delivery system on SMCs. Also, the cytotoxicity of the PEGDA and PNIPA polymers was evaluated, which are an integral part of composite nanoparticle system. The results showed there was no significant decrease in cell survival when hydrogels were photopolymerized with 0.015% (w/v) Irgacure 2959, which the earlier results had also shown to be biocompatible. For composite nanoparticle hydrogel networks photopolymerized with 0.15% (w/v) Irgacure 2959, the cell survival decreased significantly compared to the controls. From photoinitiator results, the relative cell survival for HASMCs at 0.16% (w/v) Irgacure 2959 (but without polymers) was 0.25±0.01. From FIG. 14, the relative cell survival for HASMCs exposed to media incubated composite nanoparticle hydrogel networks photopolymerized with 0.15% (w/v) Irgacure 2959 was 0.48±0.19. Therefore, these results conclude that the reactive PEGDA macromers indeed react with the free radicals and reduce the system cytotoxicity.

Studies evaluated the effects of UV dose, photoinitiator concentrations, and combined effects conclusively showed that the photoinitiator and free radicals were the most cytotoxic components. At the same time, UV light was found to not significantly affect cell survival. Additionally, ascorbic acid was shown to significantly increase cell survival, but also increased the gelation times of the hydrogel, potentially inducing cellular damage due to prolonged exposure times. Testing other antioxidants or strategies to minimize cytotoxicity and optimizing the composite nanoparticle hydrogel system by altering various components in the system such as molecular weights of PEG and concentrations of nanoparticles may be done.

### EXAMPLE 2

### Medical Applications

The applications of the invention are for controlled and sustained drug delivery systems, hypothennia, wound dressings, and tissue engineering. The commercial applications of double layer hydrogels would be: controlled and sustained drug delivery to treat cardiovascular diseases and cancer as well as to improve wound healing. With applications mentioned earlier, there will be a number of pharmaceutical, bioengineering, and biomedical companies, homeland security, department of defense and many more agencies interested in this innovation.

Present drug delivery system is not specific and causes side effects. In contrast, the composite nanoparticle hydrogel system is specific that can be photopolymerized at the site using UV light and drug can be released over a longer period of time at that site. Present techniques to prevent restenosis are thrombogenic. In addition, bare metal stents have the problem of biocompatibility. The stents coated by drug or drug loaded polymer drastically reduce the chances of restenosis, but they delay the occurrence of restenosis and larger the risk of late stent thrombosis. In the composite nanoparticle hydrogel system, stimulated drug release is achieved using thermosensitive PNIPA-AAm nanoparticles. Due to composite network and outer layer of PEGDA the drug released from PNIPA-AAm nanoparticles slowly diffuses through these PEGDA barriers, extending the period of drug release. Consequently, the risk of late stent thrombosis can be avoided by the sustained drug release and having biocompatible material.

Present wound healing techniques involve films, foams, hydrocolloids, and hydrogels. Films exhibit limited or no absorption of the wound fluids; foams can dry the wound in absence of sufficient exudate, whereas hydrocolloids can break down in the wound and residue removal requires a lot of time. In contrast, the composite nanoparticle hydrogel system is water based dressings which can be photopolymerized at the wound in a short period of time. The composite nanoparticle hydrogel network maintains a moist environment that facilitates the wound healing and is simple to apply and remove, allowing greater comfort and provides a moist environment which promotes the cell migration. Also, this composite nanoparticle hydrogel device can allow the ability to control thermoresponsive drug release very effectively as surface wounds can be heated above LCST easily by physician, thereby facilitating drug release.

### EXAMPLE 3

### Immobilization of Adhesion Peptides

The immobilization of adhesion peptides such as RGD (Arg-Gly-Asp sequence, SEQ ID NO: 1) into the composite nanoparticle hydrogel network may promote cell adhesion and cell growth. The cell adhesion ligand, RGD, will be incorporated into the PEG hydrogel network. In brief, a 10% molar excess of NH₂-Tyr-Arg-Gly-Asp-Ser-COOH (YRGDS, SEQ ID NO: 2) (1 mg/ml) will be reacted with acryloyl-PEG-N-hydroxysuccinimide (ACR-PEG-NHS) (3,400 Da, Nektar) (10 mg/ml) in 50 mM of bicarbonate buffer (pH=8.4) for 2 h. The final product, ACR-PEG-RGD will be dialyzed overnight and lyophilized for 24 h. A fluoraldehyde assay (Pierce) will be used to assess indirectly the efficiency of the reaction by measuring the amount of unreacted primary amines, which is often greater than 85%. ACR-PEG-RGD (0, 0.1, 0.4, and 0.8 mM) will be added to the precursor solution containing PEGDA and PNIPA-AAm nanoparticles with Irgacure 2959 and polymerized as described above.

Human dermal fibroblasts (HDFs) and human aortic smooth muscle cells (HASMCs) will be obtained from Invitrogen. HDFs and HASMCs will be maintained on Dulbecco's modified Eagle medium (DMEM) supplemented with 10% fetal bovine serum (FBS), 1% penicillin-streptomycin. Cells will be incubated and grown at 37° C. in a 5% CO₂ environment. Cells will be seeded on the hydrogel network for a certain period (1, 3, and 6 days). The cell adhesion and proliferation will be assessed by standard bioassays such as PicoGreen and/or immunostaining including Live/Death staining of seeded cells.

What is claimed is:

1. A composite nanoparticle hydrogel network comprising a plurality of poly(N-isopropylacrylamide-co-acrylamide) nanoparticles uniformly embedded in a plurality of cross-linked poly(ethylene glycol) diacrylate monomers forming a composite nanoparticle hydrogel network, wherein the poly(N-isopropylacrylamide-co-acrylamide) nanoparticles are temperature sensitive and include a lower critical solution temperature, such that when the local temperature is increased to or above the lower critical solution temperature, the poly(N-isopropylacrylamide-co-acrylamide) nanoparticles undergoes a reversible phase transition to expel a bioactive molecule through the cross-linked poly(ethylene glycol) diacrylate monomers at a release rate.

2. The composite of claim 1, wherein the release rate further comprises a triphasic release, including a first initial burst release, a second sustained burst release, and a third plateau release.

3. The composition of claim 1, wherein the initial burst release equals 673.27+(163.78)M−(108.51)P−(161.21)N+(1.21)T−(4.06)MT+(2.21)PT+(12.75)NT; wherein M=PEGDA molecular weight, P=PEGDA concentration, T=temperature, N=PNIPA-AAm nanoparticle concentration, MT is the combined effect of PEGDA MW and temperature, PT is the combined effect of PEGDA concentration and temperature, and NT is the combined effect of PNIPA-AAm nanoparticle concentration and temperature.

4. The composite of claim 1, wherein the second sustained burst release equals 464.34+(19.03)M−(26.37)P−(62.58)N−(7.55)T−(0.4)MT+(0.37)PT+(3.52)NT, wherein M=PEGDA molecular weight, P=PEGDA concentration, T=temperature, N=PNIPA-AAm nanoparticle concentration, MT is the combined effect of PEGDA MW and temperature, PT is the combined effect of PEGDA concentration and temperature, and NT is the combined effect of PNIPA-AAm nanoparticle concentration and temperature.

5. The composite of claim 1, wherein the third plateau release equals −17.74+(2.75)M+(0.27)P+(4.12)N+(0.6)T−(0.06)MT−(0.0072)PT−(0.097)NT, wherein M=PEGDA molecular weight, P=PEGDA concentration, T=temperature, N=PNIPA-AAm nanoparticle concentration, MT is the combined effect of PEGDA MW and temperature, PT is the combined effect of PEGDA concentration and temperature, and NT is the combined effect of PNIPA-AAm nanoparticle concentration and temperature.

6. The composite of claim 1, wherein the cross-linked poly(ethylene glycol) diacrylate monomers include a hydrogel swelling ratio according to the equation
$$S . R . = \frac{{W}_{S} - {W}_{D}}{{W}_{D}} ;$$
wherein (W_{S}) is the swollen weight and (W_{D}) is the dry weight of the composite nanoparticle hydrogel network.

7. The composite of claim 1, further comprising an outer layer cross-linked poly(ethylene glycol) diacrylate monomers surrounding the composite nanoparticle hydrogel network.

8. A process of forming a composite nanoparticle hydrogel network comprising:
a. dispersing poly(N-isopropylacrylamide-co-acrylamide) nanoparticles in deionized water with a bioactive molecule to form a stock suspension, wherein the poly(N-isopropylacrylamide-co-acrylamide) nanoparticles include a concentration;
b. dissolving poly(ethylene glycol)diacrylate including a concentration in the stock suspension with a photoinitiator stock solution, wherein the photoinitiator includes a concentration; and
c. exposing the photoinitiator stock solution to ultraviolet at a rate and a period of time to form a composite nanoparticle hydrogel network.

9. The process of claim 8, wherein the poly(ethylene glycol) diacrylate includes a molecular weight of 3.4 kDa or 8 kDa.

10. The process of claim 8, where the poly(N-isopropylacrylamide-co-acrylamide) nanoparticles includes a concentration between about 10 to 15% w/v.

11. The process of claim 8, wherein the poly(N-isopropylacrylamide-co-acrylamide) nanoparticles includes a concentration between about 2 to 4% w/v in the stock suspension.

12. The process of claim 8, wherein the dissolving step includes a temperature of about 23° C.

13. The process of claim 8, wherein the photoinitiator solution includes an antioxidant.

14. The process of claim 8, wherein the exposing step includes the rate and period of time for biocompatibility.

15. The process of claim 8, further comprising immersing the composite nanoparticle hydrogel network in a second solution containing poly(ethylene glycol)diacrylate and a photoinitiator, and exposing the second solution to ultraviolet light to form an outer layer cross-linked layer of poly(ethylene glycol)diacrylate.

16. A composite nanoparticle hydrogel system comprising a precursor solution including of the PNIPA-acrylamide (PNIPA-AAm) nanoparticles loaded with a bioactive molecule, a plurality of photoinitiators, and a plurality of PEGDA monomers, wherein exposing the precursor solution to ultraviolet (UV) light forms a hydrogel network uniformily entrapping the PNIPA-AAm nanoparticles to form a protective barrier.

17. The system of claim 16, wherein the photoinitiator solution includes a concentration to increase cell survival.

18. The system of claim 16, wherein the precursor solution includes an antioxidant.

19. The system of claim 16, further comprising an immobilized adhesion peptide incorporated with the hydrogel network.
